(19) United States
(12) Patent Application Publication
(10) Pub. No.: US 2006/0276400 A1
(43) Pub. Date: Dec. 7, 2006

(54) Modulation of cholesteryl ester transfer protein (CETP) activity

(76) Inventors: Hedy Adari, Sudbury, MA (US); Lawrence Thomas, Easton, MA (US); Charles W. Ritterhaus, Malden, MA (US)

Correspondence Address: LEON R. YANKWICH;YANKWICH & ASSOCIATES, 201 BROADWAY, CAMBRIDGE, MA, 02139, US

(21) Appl. No.: 11/145,783

(22) Filed: Jun. 6, 2005

(51) Int. Cl. A61K 38/17; A61K 38/10; C07K 7/08

(52) U.S. Cl. 514/13; 530/326; 530/327; 530/328

(57) ABSTRACT

The present invention relates to autoantigenic vaccine peptides comprising a universal helper T cell epitope portion linked to a B cell epitope portion from the N-terminus of cholesteryl ester transfer protein (CETP). The vaccine peptides are useful for eliciting an autoimmune response in a vaccinated individual, i.e., raising antibodies against the individual's endogenous CETP, in turn modulating circulating CETP activity, reducing LDL-cholesterol levels, and increasing HDL-cholesterol levels, which in turn is helpful to treat cardiovascular disease, such as atherosclerosis.

### FIELD OF THE INVENTION

This invention relates to hybrid vaccine peptides comprising a helper T cell epitope portion and a B cell epitope-portion derived from the N-terminal region of cholesteryl ester transfer protein (CETP), which vaccine peptides are useful for eliciting an autoimmune response in an individual administered such a peptide against endogenous CETP activity. Control of CETP activity is beneficial in preventing or treating cardiovascular disease, such as atherosclerosis.

### BACKGROUND

A promising new field of research has opened in the area of treatment and prevention of cardiovascular disease and in particular the prevention and treatment of atherosclerosis by directly controlling the activity of cholesteryl ester transfer protein (CETP). Human CETP is a hydrophobic glycoprotein having 476 amino acids and a molecular weight of approximately 66,000 to 74,000 daltons. (Hesler et al., J. Biol. Chem., 262: 2275-2282 (1987)). CETP mediates the transfer of cholesteryl esters from high density lipoproteins (HDL) to triglyceride (TG)-rich lipoproteins such as low density lipoproteins (LDL) and very low density lipoproteins (VLDL), and also the reciprocal exchange of TG from VLDL to HDL (Hesler et al., id.). The region of CETP defined by the carboxyl-terminal 26 amino acids, and in particular amino acids 470-475, has been shown to be especially important for neutral lipid binding involved in neutral lipid transfer. (Hesler et al, J. Biol. Chem., 263: 5020-5023 (1988)). CETP may play a role in modulating the levels of cholesteryl esters and TG associated with various classes of lipoproteins. A high CETP cholesteryl ester transfer activity has been correlated with increased levels of LDL-associated cholesterol and VLDL-associated cholesterol, which in turn are correlated with increased risk of cardiovascular disease (see, e.g., Tato et al., Arterioscler. Thromb. Vascular Biol., 15: 112-120(1995)).

Decreased susceptibility to cardiovascular disease, such as atherosclerosis, is generally correlated with increased absolute levels of circulating HDL-cholesterol (or HDLc, so-called “good cholesterol”) and also increased levels of HDLc relative to circulating levels of LDL-cholesterol (LDLc, so-called “bad cholesterol”). See, e.g., Castelli et al., J. Am. Med. Assoc., 256: 2835-2838 (1986).

Therefore, inhibition of endogenous CETP activity is an attractive therapeutic method for modulating the relative levels of lipoproteins, which, in turn, is effective for preventing the progression of or inducing regression of cardiovascular diseases, such as atherosclerosis, by increasing the ratio of circulating HDLc:LDLc in the bloodstream.

U.S. Pat. No. 6,410,022 and U.S. Pat. No. 6,284,533 describe antigenic vaccine peptides and plasmid-based vaccines, respectively, for use in the modulation or inhibition of CETP activity for the treatment or prevention of atherosclerosis. The disclosed vaccine peptides are comprised of a universal helper T cell epitope peptide linked to a B cell epitope-containing peptide from CETP. When administered to a mammal, the vaccine peptides cause an antibody response that provides native antibodies recognizing the mammal's own endogenous CETP, in turn causing a decrease in CETP activity. Data presented in these patents shows that use of a vaccine peptide including a B cell epitope from the C-terminal portion of CETP (i.e., amino acids 461-476 of human CETP) led to an autoimmune response producing anti-native CETP antibodies, a rise in HDL to LDL/VLDL ratio, a lowered level of circulating cholesterol, and a significant reduction in the development of atherosclerotic lesions in the arteries of test animals vaccinated with the vaccine peptide.

The foregoing developments are very promising for the development of an alternative approach to statin drugs for controlling cholesterol metabolism and therapeutically addressing cardiovascular disease. Other B cell epitopes upstream of the C-terminal sixteen amino acids of human CETP have been indicated in various studies (see, e.g., Swenson et al., J. Biol. Chem., 264(24): 14318-14326 (1989)), however no data have appeared showing another hybrid CETP B cell epitope/universal helper T cell epitope vaccine peptide that is effective for eliciting an immune response in a vaccinated individual that leads to the production of antibodies capable of neutralizing the lipid transfer activity of the individual's native, endogenous CETP. Therefore, the need still exists for the development of improved vaccine peptides or vaccine peptides that might be used as alternative or supplemental vaccines for the treatment of non-responders or low responders to previously reported vaccine peptides targeting CETP activity.

### SUMMARY OF THE INVENTION

As disclosed in the present application, it has now been surprisingly discovered that a vaccine peptide comprising a B cell epitope from the N-terminal portion of the CETP molecule, when linked to a universal helper T cell epitope, provides an autoantigenic vaccine peptide that causes an anti-endogenous CETP response, leading to reduction in CETP activity in a mammalian subject receiving the vaccine peptide. The vaccine peptides of the present invention, when administered to a mammal, are effective to decrease the activity levels of CETP in the bloodstream and increase the levels of circulating HDL-cholesterol and are therefore also useful in the treatment of cardiovascular disease, in particular atherosclerosis. The vaccine peptides of this invention, utilizing B cell epitope portions from the N-terminal region of CETP, have comparable or even superior autoimmunization activity to previously reported vaccine peptides utilizing B cell epitope portions of the C-terminal region of CETP, which is somewhat surprising, since the importance of the C-terminal region in CETP-mediated neutral lipid binding and transfer has been documented. (See, Hesler et al., J. Biol. Chem., 263: 5020-5023 (1988).) The present invention provides compositions and methods useful for the inhibition of cholesteryl ester transfer protein (CETP) activity. In particular, vaccine peptides are described which, when administered to a mammal, raise an antibody response against the mammal's own endogenous CETP, resulting in a decrease in overall CETP activity, and/or an increase in serum HDLc levels, and/or a decrease in the level of circulating cholesterol, and/or a decrease in serum LDLc or VLDLc levels in the subject administered the vaccine. These vaccine peptides are useful for the treatment of atherosclerosis, as they are believed to inhibit the development of atherosclerotic lesions in the arteries of a subject vaccinated with the vaccine peptides.

Such vaccine peptides are comprised of a CETP B cell epitope portion and a universal (or “broad range”) helper T cell epitope portion. The B cell epitope portion is comprised of 6 to 21 consecutive amino acids of the N-terminal 21 amino acids of CETP, preferably human CETP; the universal helper T cell epitope portion is comprised of a universal immunogenic helper T cell epitope, which binds the antigen presenting site of multiple class II major histocompatability complex (MHC) molecules. The B cell epitope portion and the universal helper T cell epitope portion are linked together, preferably covalently linked, most preferably linked by a peptide or amide bond to form a fusion peptide. Preferred fusion peptides will have the B cell epitope portion linked upstream (N-terminal) to the universal helper T cell epitope portion, however the reverse arrangement is also contemplated. Multimers, especially dimers, of the vaccine peptides of this invention are also contemplated.

In a preferred embodiment, a universal helper T cell epitope portion of a vaccine peptide of this invention is an immunogenic segment of amino acids such as short segments of tetanus toxin or diptheria toxin, or immunogenic peptides known from pertussis vaccine, Bacile Calmette-Guerin (BCG), polio vaccine, measles vaccine, mumps vaccine, rubella vaccine, and purified protein derivative (PPD) of tuberculin. An immunogenic carrier protein such as keyhole limpet hemocyanin (KLH) also may be used. Furthermore, various universal helper T cell epitopes may be linked to one another to form multiple universal helper T cell epitope portions of the vaccine peptides of this invention. In addition to naturally occurring universal helper T cell epitopes, designed peptide epitopes composed either entirely of natural amino acids or peptides comprised of a combination of natural amino acids and non-natural or synthetically modified amino acid residues may be used. Such non-natural universal helper T cell epitopes include, e.g., pan-DR epitope peptides such as those known by the PADRE™ designation (see, Alexander et al., Immunity, 1:751-762 (1994)).

In a preferred embodiment of a vaccine peptide of this invention, a universal helper T cell epitope from tetanus toxin having the sequence QYIKANSKFIGITE (SEQ ID NO: 1) is covalently linked to the C-terminus of the B cell epitope portion, the B cell epitope portion comprising a peptide having the sequence of the 21 amino-terminal amino acids of human CETP, i.e., CSKGTSHEAGIVCRITKPALL (SEQ ID NO: 2). The tetanus toxin segment may include a terminal cysteine residue to allow for dimerization of the peptide. More preferably, the vaccine peptide according to the present invention comprises the amino acid sequence from tetanus toxin QYIKANSKFIGITE (SEQ ID NO: 1) linked, preferably covalently, to amino acids 2-21 from the N-terminus of the CETP protein, i.e., SKGTSHEAGIVCRITKPALL (SEQ ID NO:3), wherein the N-terminal cysteine residue of human CETP has been removed. The most preferred embodiments of the peptides of the present invention are fusion peptides comprising a B cell epitope portion and a universal helper T cell epitope portion comprising either of the following sequences: CSKGTSHEAGIVCRITKPALLQYIKANSKFIGITE (SEQ ID NO: 4) and SKGTSHEAGIVCRITKPALLQYIKANSKFIGITE (SEQ ID NO: 5). Alternative embodiments may employ shorter spans, such as 6-8 consecutive amino acids, of the CETP N-terminal twenty-one amino acids (SEQ ID NO:2). Alternative embodiments utilizing other universal helper T cell epitope portions will include, for example, vaccine peptides incorporating such PADRE™ peptides as X₁KX₂VAAWTLKAX₁ (SEQ ID NO:42), X₁KX₂VAAWTLKAAX₁ (SEQ ID NO:48), or AKX₂VAAWTLKAAA (SEQ ID NO:49), wherein X₁ is D-Ala and X₂ is cyclohexylalanine.

Vaccine peptides of the present invention were demonstrated to reduce the level of CETP activity and increase the levels of serum HDLc in both rabbits and human CETP transgenic mice administered the peptides. Thus, the vaccine peptides according to the present invention are useful in the treatment of cardiovascular disease, such as atherosclerosis.

The vaccine peptides of this invention may be used alone or in combination with a pharmaceutically acceptable adjuvant for administration to a mammal. After an initial immunization, additional or “booster” administrations of the vaccine peptides according to the invention may advantageously be made, for example in order to achieve or maintain a beneficial anti-endogenous CETP antibody titer. The vaccine peptides disclosed herein may also be co-administered with vaccine peptides having a similar structure including a universal helper T cell epitope portion and a CETP B cell epitope portion, wherein the B cell epitope portion corresponds to a segment of CETP other than the N-terminal region of CETP (for example, the C-terminal region involved in neutral lipid binding; see, U.S. Pat. No. 6,410,022).

The present invention also contemplates a DNA plasmid-based vaccine comprising a plasmid DNA molecule including a DNA sequence encoding an autoantigenic fusion polypeptide that, when administered to a mammalian (preferably human) subject, will induce the production of autoantibodies specifically reactive with the subject's endogenous CETP. Such autoantibodies inhibit endogenous CETP activity or remove CETP from circulation, promote the formation and maintenance of an antiatherogenic serum lipoprotein profile (for example, increased HDLc levels, decreased LDLc levels, or decreased circulating cholesterol levels), and/or inhibit the development of atherosclerotic lesions in the vaccinated subject.

The DNA plasmid-based vaccine of the present invention is comprised of a synthetic gene encoding a vaccine peptide fusion protein wherein a DNA segment encoding at least one CETP B cell epitope portion is linked in-frame with a DNA segment encoding a universal helper T cell epitope portion. The synthetic gene is operably linked to suitable DNA expression control sequences for expression of the synthetic gene product in mammalian cells.

The B cell epitope portion-encoding segment of the synthetic gene of the DNA plasmid-based vaccine of the present invention is comprised of a DNA sequence encoding 6 to 21 consecutive amino acids of the amino-terminal 21 amino acids of CETP, preferably human CETP. In a preferred embodiment, the B cell epitope portion-encoding segment of the synthetic gene is comprised of a nucleotide sequence encoding the N-terminal 21 amino acids of mature human CETP or encoding amino acids 2-21 of the N-terminal 21 amino acids of mature human CETP.

The preferred DNA plasmid-based vaccine of the present invention includes the following nucleotide sequence encoding a universal helper T cell epitope portion: 5′-CAGTACATCAAGGCCAATAGCAAGTTCATCGGCATTACCGAG-3′ (SEQ ID NO: 6).

A preferred DNA plasmid-based vaccine includes the following nucleotide sequence encoding a CETP B cell epitope useful in the present invention: 5′-TGTAGCAAGGGCACCTCTCACGAGGCCGGCATCGTGTGCCGGATCACCAAAC CTGCCCTCCTG-3′ (SEQ ID NO: 45). Underlined bases represent silent nucleotide substitutions (i.e., not changing the encoded amino acid) as compared with the native mature human CETP nucleotide sequence (SEQ ID NO: 43). The substitutions are optimized for human codon usage, which should result in optimal expression levels in vivo in human cells.

Another preferred DNA plasmid-based vaccine of the present invention includes the nucleotide sequence encoding a CETP B cell epitope comprising amino acids 2-21 (SEQ ID NO: 3) of the mature human CETP: 5′-AGCAAGGGCACCTCTCACGAGGCCGGCATCGTGTGCCGGATCACCAAACCT GCCCTCCTG-3′ (SEQ ID NO: 46). Underlined bases represent silent nucleotide substitutions (i.e., not changing the encoded amino acid) as compared with the native mature human CETP nucleotide sequence (SEQ ID NO: 43). The substitutions are optimized for human codon usage.

A preferred nucleotide sequence encoding a universal helper T cell epitope and a CETP B cell epitope fusion peptide for use in the DNA plasmid-based vaccine of the present invention includes the sequence: 5′-TGTAGCAAGGGCACCTCTCACGAGGCCGGCATCGTGTGCCGGATCACCAAAC CTGCCCTCCTGCAGTACATCAAGGCCAATAGCAAGTTCATCGGCATTACCGAG-3′ (SEQ ID NO: 9). Underlined bases represent silent nucleotide substitutions (i.e., not changing the encoded amino acid) as compared with the native mature human CETP nucleotide sequence (SEQ ID NO: 43). The substitutions are optimized for human codon usage.

A further preferred nucleotide sequence encoding a universal helper T cell epitope and a CETP B cell epitope fusion peptide for use in a DNA plasmid-based vaccine of the present invention includes the nucleotide sequence encoding amino acids 2-21 from the N-terminal region of the full-length mature human CETP protein: 5′-AGCAAGGGCACCTCTCACGAGGCCGGCATCGTGTGCCGGATCACCAAACC TGCCCTCCTGCAGTACATCAAGGCCAATAGCAAGTTCATCGGCATTACCGAG-3′ (SEQ ID NO: 47). Underlined bases represent silent nucleotide substitutions (i.e., not changing the encoded amino acid) as compared with the native full-length mature human CETP nucleotide sequence (SEQ ID NO: 43). The substitutions are optimized for human codon usage.

Plasmid-based vaccines of the invention taken up by cells are transcribed and translated to produce autoantigenic fusion peptides in vivo. Expression at sufficient levels and for a sufficient period of time exposes the autoantigenic fusion peptide to the host immune system, which elicits production of autoantibodies that react specifically with endogenous CETP of the host and that serve to inhibit CETP-mediated hypercholesterolemia. This, in turn, promotes an an antiatherogenic serum lipoprotein profile and inhibits the deposit of atherosclerotic plaque in the lumen of blood vessels.

### BRIEF DESCRIPTION OF THE DRAWINGS

FIGS. 1A and 1B show rabbit anti-human CETP antibody binding data from Dot-Blot analysis for each of thirty rabbit CETP 21-mer fragments and one C-terminal 16-mer fragment (SEQ ID NOs: 10-40), tested with anti-huCETP serum collected from eleven New Zealand White rabbits immunized with full-length human CETP (SEQ ID NO:7).

FIG. 1A shows the percentage change in CETP activity and the ratio of HDL-cholesterol (HDLC) to total cholesterol, determined for serum collected from each vaccinated rabbit at week-1 pre-injection and serum collected from each vaccinated rabbit at week 12 post-injection. The changes in CETP activity and HDLc/total cholesterol ratio in rabbits immunized with human CETP indicates that anti-huCETP antibodies generated by the immunized rabbits were cross-reactive with their native (endogenous) CETP.

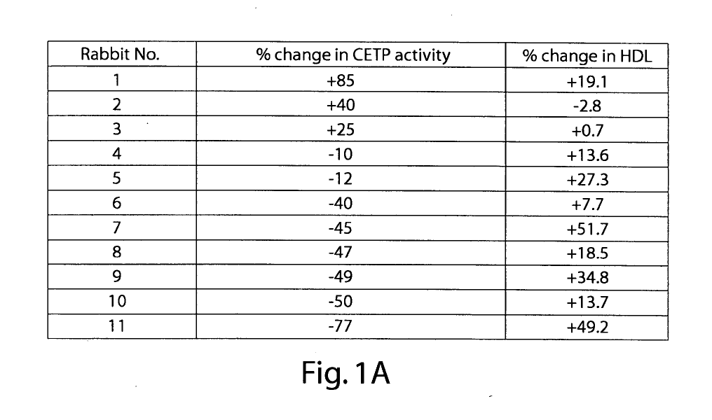

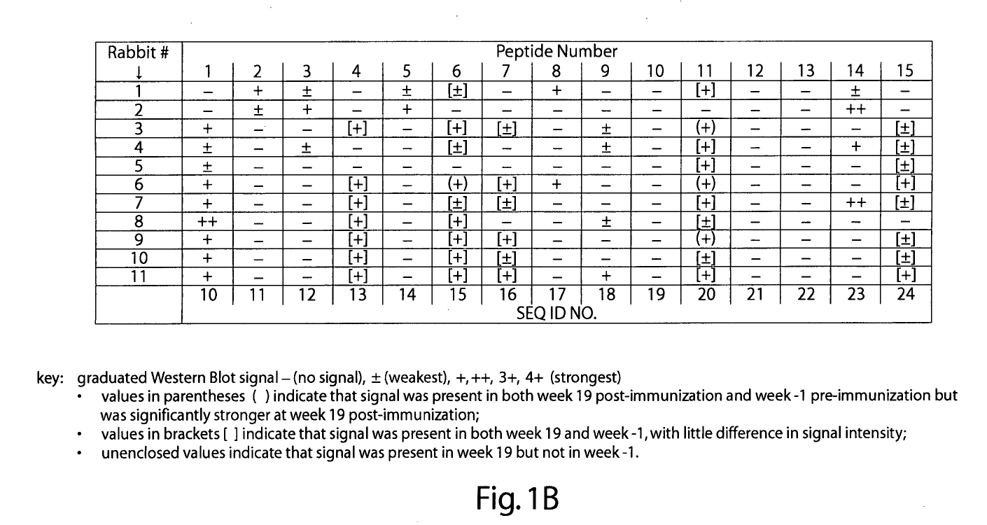

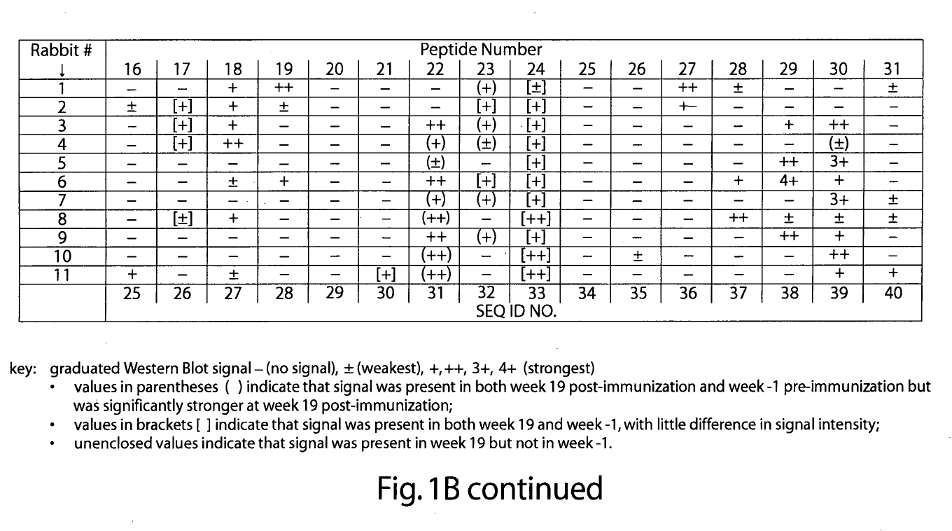

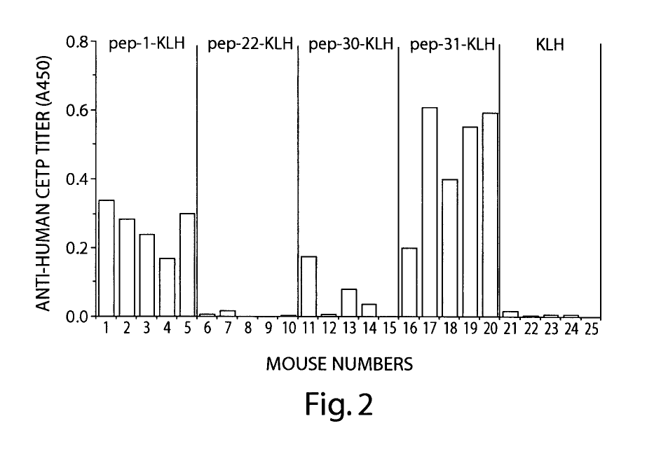

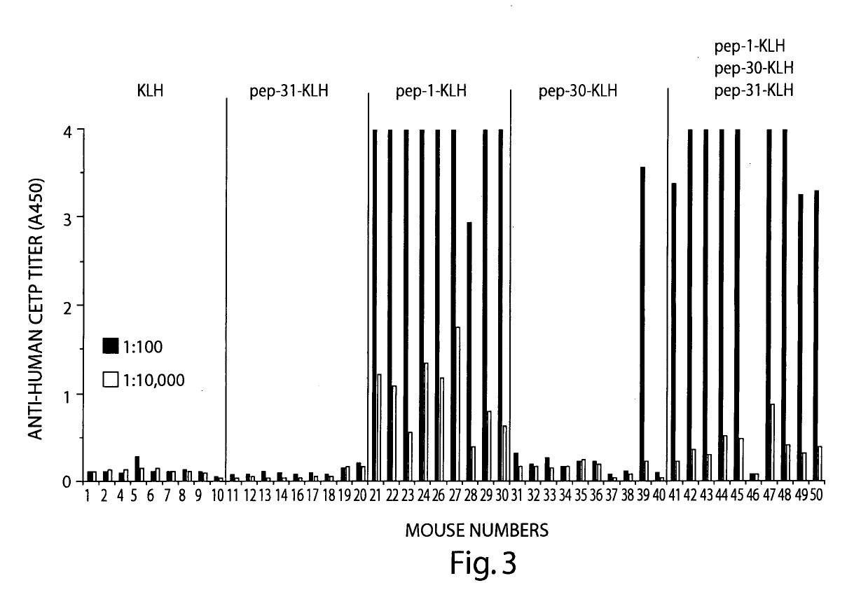

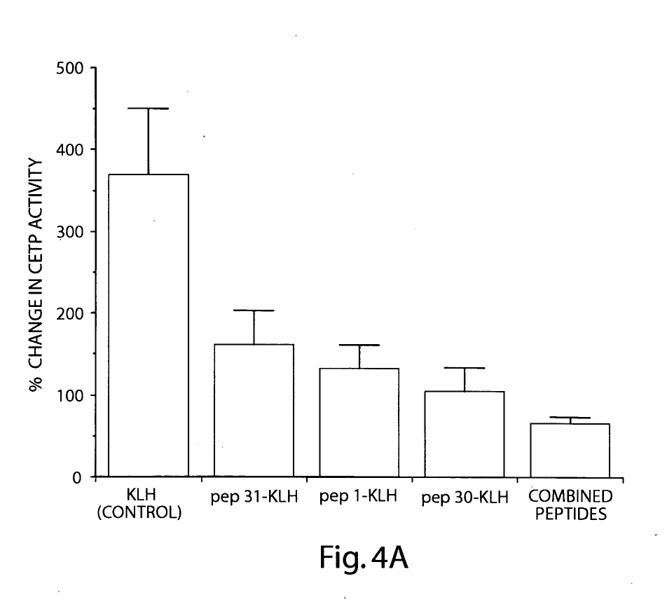

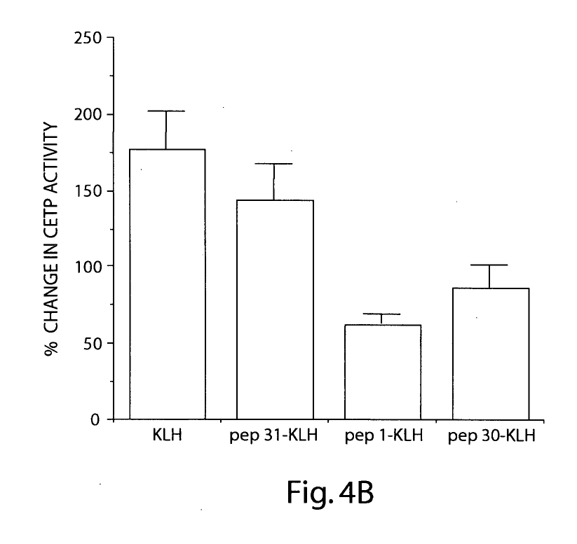

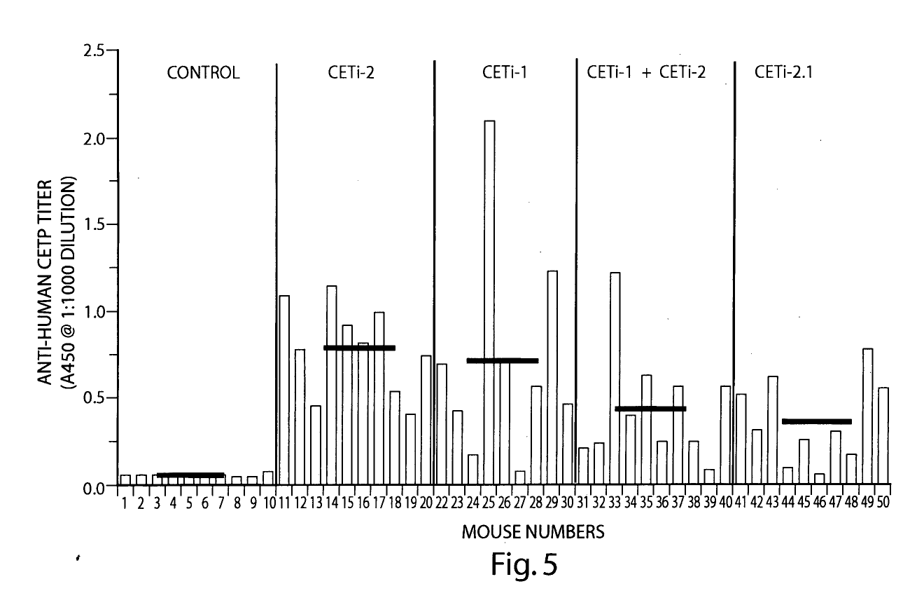

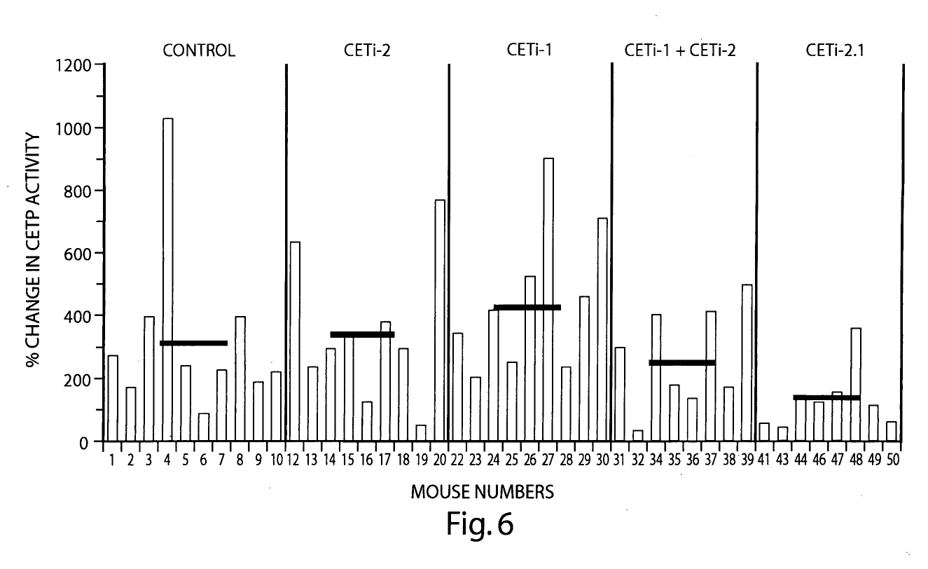

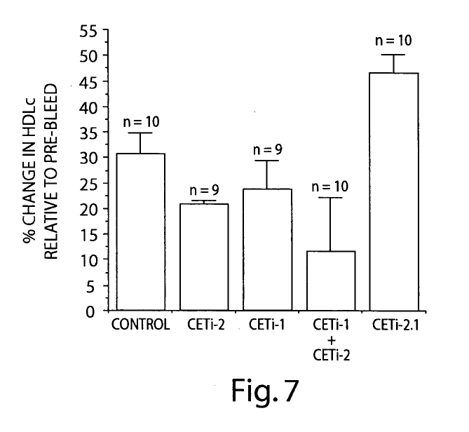

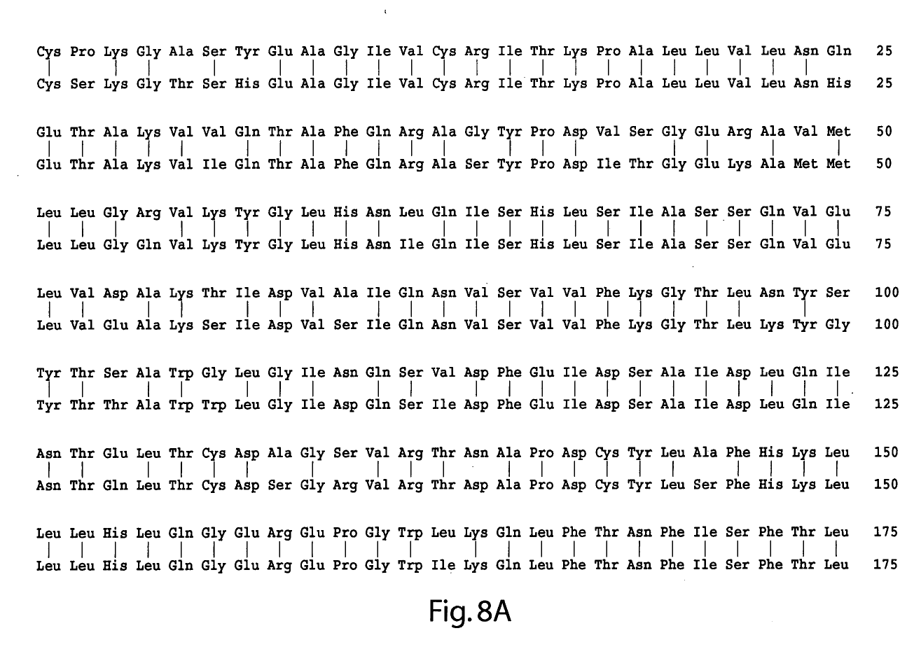

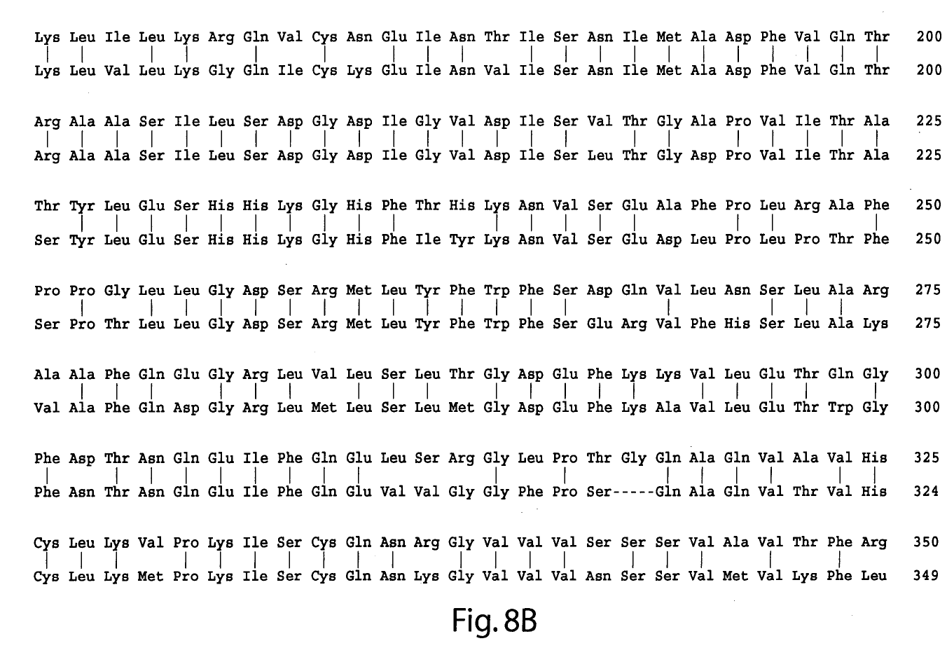

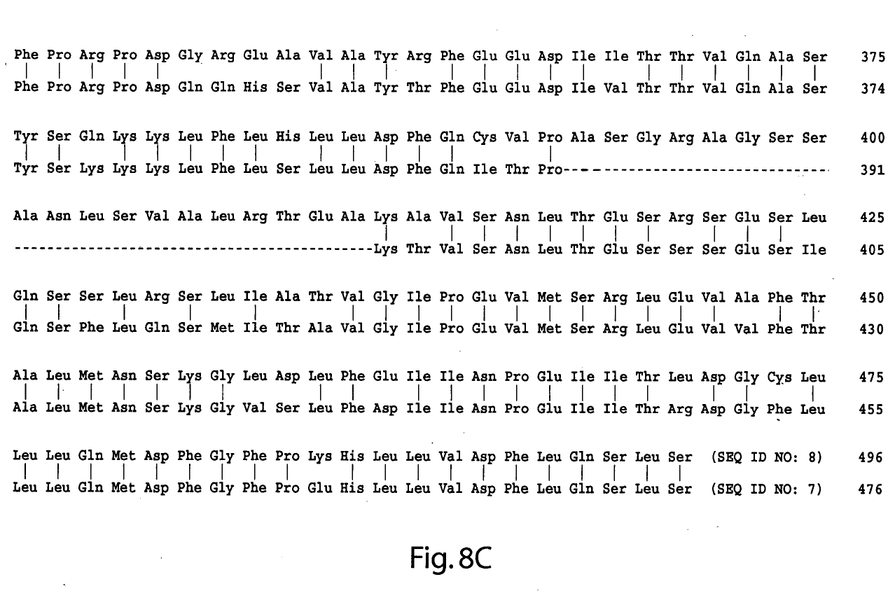

The binding data in FIG. 1B is arranged left to right beginning with data for the N-terminal 21-mer of mature rabbit CETP (peptide #1, SEQ ID NO:10), and continuing serially (with 5-amino acid overlaps) in the direction of the C terminus along the length of the rabbit CETP amino acid sequence for each 21-mer to the most C-terminal peptide, i.e., peptide #31 (SEQ ID NO:40), which is just 16 amino acids in length. As seen in FIG. 1B, the potential for any region of the full-length CETP protein to function as a B cell epitope can be immediately determined from the binding data. The results shown in FIG. 1B for each peptide indicate the strength of the signal on Dot-Blot, which was graded as no signal (−), weak (±), positive (+), strong (++), or very strong (3+ or 4+). Values were recorded in normal type where a signal was present in week 19 post-injection but not in week −1 pre-injection. Values were recorded in parentheses ( ) where a signal was present in both week 19 and week −1, but the signal was significantly higher in week 19. Finally, values were recorded in brackets [ ] where a signal was present in both week 19 and week −1, but there was no significant difference in signal intensity.

The data in FIGS. 1A and 1B are arranged vertically from the rabbit presenting the greatest increase in CETP activity between week −1 pre-injection and week 12 post-injection, i.e., +85% for rabbit #1, to the rabbit with the greatest decrease in percent CETP activity between week −1 pre-injection and week 12 post-injection, −77% for rabbit #11. In FIG. 1A, there is a correlation between an overall decrease in percent (%) CETP activity over time and an overall percent (%) increase in the HDLc/total cholesterol ratio that reflects the decrease in CETP activity. It is believed that the increase in the percent CETP activity and the increase in the percent HDLc/total cholesterol observed for rabbit #1 is an anomalous result that could be an indication of a viral or bacterial infection in that rabbit at the time of serum collection, or a result of that particular rabbit's genetic makeup, both of which factors are known to cause an increase in HDLc levels in these mammals.

FIGS. 1A and 1B considered together show the correlation between the pattern of immune reactivity for particular regions of CETP with the change in CETP activity and HDLc/total cholesterol ratio levels post-vaccination.

FIG. 2 shows the antibody titer at a 1:10 dilution of wild-type BALB-c mice receiving one priming injection and two booster injections of KLH-conjugated human CETP peptide nos. 1, 22, 30, or 31 (see Table 2, infra), the conjugates being designated pep-1-KLH, pep-22-KLH, pep-30-KLH, and pep-31-KLH, resepectively, and formulated in either Complete Freund's Adjuvant (CFA) for the prime injection, or Incomplete Freund's Adjuvant (IFA) for the booster injection(s). Control mice were administered KLH alone in CFA or IFA. Each group was comprised of 5 mice.

FIG. 3 shows the antibody titer at a 1:100 or 1:10,000 dilution for human CETP transgenic mice receiving one priming injection and two booster injections of KLH-conjugated human CETP peptides 1, 30, 31, (see, Table 2, infra), the conjugates being designated pep-1-KLH, pep-30-KLH, and pep-31-KLH, resepectively, or a combination of all three peptide conjugates, formulated in Complete Freund's Adjuvant for the prime injection and Incomplete Freund's Adjuvant for the booster injections. Control mice received KLH alone in CFA or IFA. Each group included either 9 or 10 transgenic mice.

FIGS. 4A and 4B show the percent change in CETP activity for the human CETP transgenic mice after receiving one priming injection of KLH-conjugated human CETP peptides 1, 30, or 31 (see Table 2, infra) formulated in Complete Freund's Adjuvant and a first booster injection (FIG. 4A; one week after priming injection) or a second booster injection (FIG. 4B; 13 weeks after priming injection) of the KLH-conjugated peptides formulated in Incomplete Freund's Adjuvant. Control mice received KLH alone in CFA or IFA. CETP levels are calculated relative to prebleed (pre-vaccination) levels. FIG. 4A shows the changes in CETP activity 5 weeks after the first booster dose (before the second booster), KLH-conjugated peptides 1, 30, and 31, reduced the levels of CETP activity by approximately 67%, 70%, and 56%, respectively, relative to control mice injected with KLH alone. A combination of the three conjugates reduced CETP activity in transgenic mice by approximately 82% relative to control mice receiving KLH alone.

FIG. 4B shows the percentage change in CETP activity in human CETP transgenic mice after receiving the second booster injection of KLH-conjugated peptides 1, 30, or 31. KLH injected alone was used as a control. KLH and KLH-peptides were administered (by subcutaneous injection) with CFA or IFA. CETP levels are presented relative to prebleed levels. As seen in FIG. 4B, the peptide 1 conjugate reduced CETP activity by approximately 65% relative to mice injected with KLH alone, while the peptide 30 and 31 conjugates reduced the activity by about 52% and 22%, respectively, compared to transgenic mice injected with KLH alone.

FIG. 5 shows the antibody titer of human CETP transgenic mice after receiving one priming injection and two booster injections of fusion peptides having a broad range helper T cell epitope derived from tetanus toxin, linked to selected CETP peptides: i.e., fusion peptide designated CETi-1 (SEQ ID NO:41), fusion peptide designated CETi-2 (SEQ ID NO:4), and fusion peptide designated CETi-2.1 (SEQ ID NO:5). A combination of CETi-1 and CETi-2 was administered to one of the groups. Control mice were injected subcutaneously with either CFA (prime injection) or IFA (booster injections) alone. Horizontal bars show the overall average of antibody levels for each group of mice.

FIG. 6 shows the percent change in CETP activity in human CETP transgenic mice after receiving one priming injection formulated in CFA and two booster injections formulated in IFA of a positive control vaccine peptide CETi-1 (SEQ ID NO:41), a vaccine peptide CETi-2 according to the invention (SEQ ID NO:4), a vaccine peptide CETi-2.1 according to the invention (SEQ ID NO:5), or a combination of CETi-1 and CETi-2. Control mice were injected subcutaneously with CFA (prime) or IFA (booster) alone. All CETP levels are calculated relative to prebleed levels. The serum samples are from the same experiment presented in FIG. 5. As seen in FIG. 6, CETi-2.1 reduced CETP activity levels in the transgenic mice by approximately 60% relative to control levels.

FIG. 7 shows the percent change in HDLc levels in human CETP transgenic mice after receiving one priming injection formulated in CFA and two booster injections formulated in IFA of a positive control vaccine peptide CETi-1 (SEQ ID NO:41), a vaccine peptide CETi-2 according to the invention (SEQ ID NO:4), a vaccine peptide CETi-2.1 according to the invention (SEQ ID NO:5), or a combination of CETi-1 and CETi-2 (CETi-1+2). Control mice were injected subcutaneously with either CFA (prime) or IFA (booster) alone. The serum samples are from the same experiment presented in FIG. 5; average values for all the mice of each group are presented. As seen in FIG. 7, administration of CETi-2.1 led to an increase in the level of HDLc of approximately 40% relative to control.

FIG. 8A-C shows an alignment of the amino acid sequences of mature rabbit CETP (SEQ ID NO: 8) with mature human CETP (SEQ ID NO: 7). The rabbit CETP is shown over the aligned human CETP sequence. The rabbit sequence includes 20 more amino acid residues than the human sequence, and the human sequence shows a 1-amino acid and a 19-amino acid gap (indicated with dashes, ---, in the human sequence) in order to show the residue matches (indicated with a vertical line, |) most clearly.

### DETAILED DESCRIPTION OF THE INVENTION

CETP has been validated as a therapeutic target for raising levels of HDL-cholesterol, raising the ratio of HDL-cholesterol to LDL-cholesterol, and for treating atherosclerosis (Davidson et al., Atherosclerosis, 169(1): 113-117 (July 2003); U.S. Pat. No. 6,410,022). This invention is directed to the modulation of endogenous CETP activity by providing CETP vaccine peptides derived from the N-terminus of the protein, or DNA based plasmid vaccines encoding the N-terminal CETP peptides, which peptides are useful for inducing an immune response in individuals against their own endogenous CETP, i.e., autoantibodies, thereby promoting an improved serum lipoprotein profile, e.g., decreasing the level of CETP activity in the bloodstream, increasing the levels of circulating HDLc or decreasing the levels of circulating LDLc/VLDLc, all of which are correlated with a decreased risk of cardiovascular disease.

The present invention provides CETP vaccine peptides, for inducing the production of anti-endogenous CETP antibodies in a vaccinated mammal, which are synthetic (non-naturally-occurring) vaccine peptides comprising a helper T cell epitope portion, comprising an amino acid sequence of a universal helper T cell epitope (see, for example, SEQ ID NO: 1), and a B cell epitope portion, comprising an amino acid sequence from the amino-terminal region of CETP, specifically from the amino-terminal 21 amino acids of CETP (see, for example, SEQ ID NO: 2 showing the N-terminal 21 amino acids from the mature full-length human CETP protein). Such CETP vaccine peptides are “autoantigenic”, that is, when administered to a mammalian subject they elicit production of specific antibodies against that peptide (antigen) which also bind that mammal's endogenous CETP, i.e., the mammal's native protein. Thus, the vaccine peptides of this invention are hybrid (universal helper T cell epitope peptide+CETP B cell epitope peptide) immunogenic moieties that have the capacity to stimulate the formation of autoantibodies which specifically bind endogenous CETP and/or inhibit endogenous CETP activity in a mammal vaccinated with the hybrid peptide or a DNA-based plasmid vaccine capable of directing in vivo expression of such a hybrid peptide.

Universal Helper T Cell Epitope Portion

The universal helper T cell epitope portion of the vaccine peptides of the present invention comprises an amino acid sequence of a universally immunogenic or “broad range” helper T cell epitope, which is defined as a peptide which can be presented by multiple major histocompatibility complex (MHC) haplotypes and thereby activate helper T cells, which, in turn, stimulate B cell growth and differentiation. Many examples of what are termed “universal” or “broad range” helper T cell epitopes which have been used for human vaccination are known in the art and include, for example, peptide segments of tetanus toxin (tt) and diptheria toxin (dt) (see, e.g., Panina-Bordignon et al., Eur. J. Immunol., 19: 2237-2242 (1989); Etlinger, H. M., Immunol. Today, 13: 52-55 (1992); Valmori, et al., J. Immunol., 149: 717-721 (1992); Talwar et al., Proc. Natl. Acad. Sci. USA, 91: 8532-8536 (1994)).

In addition to short segments of tt and dt, tetanus toxoid (i.e., formaldehyde-detoxified tetanus toxin) or diphtheria toxoid can be used as a broad range helpter T cell epitope portion. Other broad range helper T cell epitope sequences useful in this invention include immunogenic peptides known from pertussis vaccine, Bacile Calmette-Guerin (BCG), polio vaccine, measles vaccine, mumps vaccine, rubella vaccine, and purified protein derivative (PPD) of tuberculin (see, e.g., Etlinger, H. M., Immunol. Today, 13: 52-55 (1992)); incorporated herein by reference). Additional universal helper T cell epitopes include synthetic compounds known as pan-DR-binding epitope (PADRE™) peptides, such as the peptide having the formula: X₁KX₂VWANTLKAAX₁ (SEQ ID NO:42), where X₁=D-alanine and X₂=cyclohexylalanine. (Alexander et al., Immunity, 1: 751-761 (1994)). Furthermore, two or more copies of the same or various different universal or broad range helper T cell epitopes may be linked to one another to form multiple or multivalent helper T cell epitope portions of the vaccine peptides of this invention. For example, a vaccine peptide of this invention can be synthesized containing a multiple or multivalent helper T cell epitope portion comprising an amino acid sequence of a tt helper T cell epitope segment and a dt helper T cell epitope segment.

An immunogenic carrier protein may also be used as the universal helper T cell epitope portion of the vaccine peptide. Such carrier proteins are selected because they have immunostimulatory properties presumably from the presence of several helper T cell epitope sites, and also include convenient binding site(s) for covalent attachment of one or more CETP B cell epitope portions. One such immunogenic carrier protein is keyhole limpet hemocyanin (KLH). KLH contains multiple lysine residues in its amino acid sequence, and each of these lysines is a potential site at which a B cell epitope peptide or a whole vaccine peptide as described herein could be linked (for example, using maleimide-activated KLH, Catalog No. 77106, Pierce Chemical Co., Rockford, Ill.). Other immunogenic carrier proteins useful in the present invention include heat shock proteins HSP70 and HSP65 from Mycobacterium tuberculosis.

In particular embodiments, sequences of complement protein C3d may be used in addition to the universal helper T cell epitope(s), to enhance the magnitutde of the B cell response, leading to higher antibody titers.

In embodiments of the present invention, the universal helper T cell epitope portion is not a segment of CETP. During maturation, an individual's immune system generally has eliminated from the T cell repertoire any T cells capable of recognizing the individual's own (self) proteins (including CETP), which otherwise would encounter the immune system and initiate an immune response. Auto-immune responses are typically regarded as inappropriate and detrimental, and can lead to the manifestation of disease states, with potentially harmful or even fatal consequences. Since the helper T cells that might be capable of recognizing endogenous CETP epitopes normally have been eliminated from an individual's immune system, CETP would not be expected to be a source of any universal helpter T cell epitopes, and the practice of the present invention would require selection of a universal helper T cell epitope portion from a source other than CETP. Thus, the universal helper T cell epitope portion is occasionally referred to herein as a “non-CETP-related” component of the vaccine peptides of the invention. This naturally means that CETP itself, or a fragment thereof, without further modification (i.e., by linking to a universal helper T cell epitope), is not an embodiment of the present invention. Also, because the components of the vaccine peptides of this invention include CETP-related and non-CETP related components, the vaccine peptides are aptly regarded as hybrid polypeptides, combining elements from at least two different sources.

Preferred universal helper T cell epitope portions for use in vaccine peptides of this invention comprise universal immunogenic peptide fragments of tetanus toxin or diphtheria toxin. In a more preferred embodiment, the peptides of this invention use tetanus toxin segments having the amino acid sequence: QYIKANSKFIGITE (SEQ ID NO: 1), or FNNFTVSFWLRVP KVSASHLE (SEQ ID NO:50), or repeats or combinations thereof. Most preferably, the vaccine peptides of this invention utilize the universal helper T cell epitope from tetanus toxin having the amino acid sequence QYIKANSKFIGITE (SEQ ID NO:1). In addition to the various examples of universal helper T cell epitopes discussed above, additional universal helper T cell epitopes can be determined using a standard proliferation assay for MHC class II (helper) T cell epitopes (see, for example, Current Protocols in Immunology, Vol. 1 (Coligan et al., eds.) (John Wiley & Sons, Inc., New York, N.Y., 1994), pages 3.12.9-3.12.14).

CETP B Cell Epitope Portion

The B cell epitope portion of a vaccine peptide according to the present invention comprises an amino acid sequence from the amino-terminal region of CETP. Specifically, the B cell epitope portion comprises a peptide of at least six consecutive amino acids of the twenty-one N-terminal amino acids of CETP. In a preferred embodiment the B cell epitope portion of a vaccine peptide of this invention comprises the amino terminal 21 amino acids of mature human CETP protein. The full-length amino acid sequence of mature human CETP is shown in SEQ ID NO:7, the amino-terminal 21 amino acids of mature human CETP are shown in SEQ ID NO:2, the full-length amino acid sequence of rabbit CETP is shown in SEQ ID NO: 8.

More preferably, the B cell epitope portion (or “CETP-related” portion) of the vaccine peptides of this invention may be any fragment of the amino-terminal region of CETP which is at least six, preferably at least eight, consecutive amino acids from the amino-terminal 21 amino acids of CETP.

In a preferred embodiment, the CETP B cell epitope portion of a vaccine peptide according to the present invention is comprised of amino acids 2-21 of the human CETP, i.e., the N-terminal amino acid sequence without the cysteine residue located at amino acid position one of the CETP sequence (see, SEQ ID NO: 3).

The B cell epitope portion may utilize a segment of at least six consecutive amino acids from any mammalian CETP N-terminal region, however it is preferred to utilize a segment from the CETP of the same species that the vaccine peptide is intended for. For example, where the subject to be autoimmunized with the vaccine peptide is a human, and endogenous human CETP is thus the target for immunomodulation, the vaccine peptide B cell epitope portion will preferably be a segment selected from the N-terminal 21 amino acids of mature human CETP (see, SEQ ID NO: 2). Although less preferred, however, a xenogeneic N-terminal CETP segment may also be used, so long as it is effective to elicit antibodies cross-reactive with the endogenous CETP of the vaccinated subject.

Preparation of Vaccine Peptides

The universal helper T cell epitope (non-CETP-related) and the B cell epitope (CETP-related) portions of the CETP vaccine peptides of this invention are linked together to form autoantigenic moieties. The universal helper T cell epitope and B cell epitope portions may be covalently linked, directly by peptide bonds or via a cross-linking molecule. Where cross-linking molecules are used, they must join the universal or broad range helper T cell epitope portion and B cell epitope portion of the vaccine peptide together, without causing the peptide to become toxic to the vaccinated subject or significantly interfering with or reducing the overall immunogenicity of the vaccine peptide. Suitable cross-linking agents and molecules include amino acids, for example, using one or more glycine residues to form a “glycine bridge” between the universal helper T cell epitope and B cell epitope portions of the vaccine peptides of this invention; disulfide bonds between cysteine residues that exist in the universal helper T cell epitope portion and B cell epitope portion; cross-linking molecules such as glutaraldehyde (Kom et al., J. Mol. Biol., 65: 525-529 (1972)), and other bifunctional cross-linker molecules to link a universal helper T cell epitope portion to a B cell epitope portion are suitable for use in the present invention. Bifunctional cross-linker molecules possess two distinct binding sites, one of the sites can form a covalent attachment to a reactive site on the universal helper T cell epitope portion, and the other cross-linker binding site can form a covalent attachment to a reactive site on a B cell epitope portion. General methods for the use of cross-linking molecules are reviewed in Means and Feeney, Bioconjugate Chem., 1: 2-12 (1990).

Preferably, the universal helper T cell epitope and CETP B cell epitope portions of the vaccine peptides of this invention are covalently linked end-to-end to form a continuous fusion peptide. See, e.g., the synthetic peptides designated CETi-2 and CETi-2.1 (SEQ ID NOs: 4 and 5, respectively). Most preferably, a selected universal helper T cell epitope portion forms the carboxyl terminal portion of the vaccine peptide with its amino terminal amino acid residue covalently linked in a peptide bond to the carboxyl terminal amino acid of a selected CETP-related amino acid sequence (B cell epitope portion) of the vaccine peptide. (See, for example, SEQ ID NOs: 4 and 5).

The peptides of this invention can be produced by any of the available methods known in the art for synthesizing peptides of defined amino acid sequence. Direct synthesis of the peptides of the invention may be accomplished using conventional techniques, including solid-phase peptide synthesis, solution-phase synthesis, etc. Solid-phase synthesis is preferred. See, Stewart et al., Solid-Phase Peptide Synthesis (1989), W. H. Freeman Co., San Francisco; Merrifield, J. Am. Chem. Soc., 85:2149-2154 (1963); Bodanszky and Bodanszky, The Practice of Peptide Synthesis (Springer-Verlag, New York 1984), incorporated herein by reference.

Polypeptides according to the invention may also be prepared commercially by companies providing peptide synthesis as a service (e.g., BACHEM Bioscience, Inc., King of Prussia, Pa.; Quality Controlled Biochemicals, Inc., Hopkinton, Mass.).

Automated peptide synthesis machines, such as those manufactured by Perkin-Elmer Applied Biosystems, also are available.

Alternatively, the peptides of this invention may be produced using synthetic and recombinant nucleic acid technology. For example, one of ordinary skill in the art can design from the known genetic code a 5′ to 3′ nucleic acid sequence encoding a vaccine peptide of this invention. A DNA molecule including the coding sequences of the universal helper T cell epitope and CETP B cell epitope portions (and any linking peptide, such as polyglycine, or other additional residue(s), such as a C-terminal or N-terminal cysteine, if desired) can readily be synthesized either using an automated DNA synthesizer or by a commercial DNA synthesizing service. The synthesized DNA molecule can then be inserted into any of a variety of available gene expression systems (e.g., bacterial plasmids, bacteriophage expression vectors, retroviral expression vectors, baculoviral expression vectors) using standard methods available in the art (e.g., Sambrook et al., Molecular Cloning: A Laboratory Manual, Vols. 1-3 (Cold Spring Harbor Laboratory, Cold Spring harbor, N.Y., (1989)). The expressed peptide is then isolated from the expression system using standard methods to purify peptides.

The polypeptide compound is preferably purified once it has been isolated or synthesized by either chemical or recombinant techniques. For purification purposes, there are many standard methods that may be employed including reversed-phase high-pressure liquid chromatography (RP-HPLC) using an alkylated silica column such as C₄-, C₈- or C₁₈-silica. A gradient mobile phase of increasing organic content is generally used to achieve purification, for example, acetonitrile in an aqueous buffer, usually containing a small amount of trifluoroacetic acid. Ion-exchange chromatography can also be used to separate peptides based on their charge. Purification of the vaccine peptides of this invention may be expedited by employing affinity chromatography or immunoprecipitation, e.g., based on using antibodies or other ligands recognizing the particular universal helper T cell epitope portion or B cell epitope portion of the vaccine peptide to be purified. The degree of purity of the polypeptide may be determined by various methods, including identification of a major large peak on HPLC. A polypeptide that produces a single peak that is at least 95% of the input material on an HPLC column is preferred. Even more preferable is a polypeptide that produces a single peak that is at least 97%, at least 98%, at least 99% or even 99.5% or more of the input material on an HPLC column.

Uses of the Vaccine Peptides

The peptides of this invention are used as autoimmunogenic compositions that elicit production of endogenous autoantibodies which specifically bind to the immunized subject's endogenous CETP and/or modulate (i.e., decrease or inhibit) endogenous CETP activity in the immunized subject. A vaccine composition including one or more vaccine peptides of this invention may be used. For example, peptides having different universal helper T cell epitope portions (e.g., different universal helper T cell epitopes) and/or different CETP B cell epitope portions (e.g., different CETP-related portions comprised of the amino terminal 21 amino acids of CETP or fragments thereof spanning 6 or more amino acids) may be combined and administered as a single vaccine composition. In addition, vaccine peptides according to this invention may be combined with other CETP vaccine peptides, as disclosed in, for example, U.S. Pat. No. 6,410,022.

Pharmaceutically acceptable adjuvants, such as alum, may be mixed with vaccine peptides of this invention. Alum is the single adjuvant currently approved for use in administering vaccines to humans (see, Eldridge et al., In Immunobiology of Proteins and Peptides V: Vaccines: Mechanisms, Design, and Applications, Atassi, M. Z., ed. (Plenum Press, New York, 1989), page 192). Recently, alum was used in combination with a sodium phthalyl derivative of lipopolysaccharide to administer a vaccine shown to be effective against human chorionic gonadotropin to humans (see, Talwar et al., Proc. Natl. Acad. Sci. USA, 91: 8532-8536 (1994)).

Other conventional adjuvants may be used as they are approved for a particular use. For example, biodegradable microspheres comprised of poly (DL-lactide-co-glycolide) have been studied as adjuvants for oral or parenteral administration of vaccine compositions (Eldridge et al., In Immunobiology of Proteins and Peptides V. Vaccines: Mechanisms, Design, and Applications, Atassi, M. Z., ed. (Plenum Press, New York, 1989), page 192).

Other adjuvants have been used for administering vaccines to non-human mammals. For example, Complete Freund's Adjuvant (Sigma Chemical Co., St. Louis, Mo.), Incomplete Freund's Adjuvant (Sigma Chemical Co., St. Louis, Mo.), and the MPL, RC-259 and Ribi Adjuvant System (RAS) available from Corixa Corp. (Seattle, Wash.) are well known adjuvants routinely used to administer antigens to mammalian subjects, which may also eventually be approved for use in humans. In addition, adjuvant structures may also be mixed with or, preferably, covalently incorporated into peptides of this invention, for example at the amino or carboxyl terminal amino acid residue of the peptides. Such incorporated adjuvants include lipophilic N-palmitoyl-S-[2,3-bis(palmitoyloxy)propyl)]-cysteine (“Pam₃-Cys-OH”); glycopeptides such as N-acetyl-glucosaminyl-N-acetylmuramyl-alanyl-D-isoglutamine (“GMDP”), muramyl dipeptides, and alanyl-N-adamantyl-D-glutamine; and polyamide gel-based adjuvants which can easily be attached to peptides during their in vitro chemical synthesis (see, Synthetic Vaccines, Nicholson, B. H., ed. (Blackwell Scientific Publication, Cambridge, Mass., 1994), pp. 236-238).

In addition, the vaccine peptides of the present invention may be linked to other molecules that may enhance the immunogenicity of the peptides. For example, linking peptides of this invention to a surface of a larger molecule, such as serum albumin, may enhance immunogenicity because the epitopes of the vaccine peptides are presented to the immune system of an individual as adjacent multiple repeated copies (see, e.g., Tam, J. P., Proc. Natl. Acad. Sci. USA, 85: 5409-5413 (1988); Wang, C. Y., et al., Science, 254: 285-288 (1991); Marguerite, M., et al., Mol. Immunol., 29: 793-800 (1992)). Such “multiple” or “multivalent” arrangements of the vaccine peptides of this invention can be prepared using cross-linker molecules. For example, bifunctional cross-linker molecules possess two reactive sites, one of the sites can attach the linker to a vaccine peptide of this invention and the other site is available to react with a different molecule, e.g., a larger protein like serum albumin or a resin or polymeric bead. Thus, covalent cross-linker molecules may be used to link vaccine peptides to other proteins or substrates to form multicopy arrangements of the peptides (multicopy peptide assemblies). Linking vaccine peptides of this invention to another molecule or surface should be carried out in a manner that does not significantly disrupt or reduce the autoantigenic characteristics of the vaccine peptides. Preferably, the use of such linker molecules enhances the immunogenicity of the vaccine peptides of this invention as evidenced, for example, by a more rapid rise in anti-CETP antibody titer and/or production of higher affinity anti-CETP antibodies than when individuals are administered vaccine peptides that are not linked. Such cross-linker molecules may also be used to attach peptides of this invention to an “immunogenic enhancer” molecule such as granulocyte-macrophage colony-stimulating factor (GM-CSF), which has been shown to serve as an effective immunogenic enhancer in generating the production of specific anti-tumor antibodies (e.g., Tao, M. H., et al., Nature, 362: 755-758 (1993)).

The vaccine peptides of this invention may be administered, either alone or in association with one or more pharmaceutically acceptable carriers or adjuvants, in the same manner as a conventional vaccine, such as, e.g., tetanus vaccine. Suitable means include, for example, subcutaneous, intramuscular or intravenous injection. However, in contrast to conventional vaccines, which elicit an immune reaction against a non-endogenous, “foreign” antigen, such as, e.g., tetanus toxoid, the vaccine peptides of the present invention elicit an autoantibody response against endogenous CETP of the vaccine recipient. In some embodiments of this invention, the vaccine peptides may also be combined and administered with vaccines for other diseases or disorders.

The immune response elicited against endogenous CETP should significantly inhibit endogenous CETP function, in particular the transfer of cholesteryl esters from HDL to LDL/VLDL, and thereby produce a change in the circulating levels of LDLc and/or VLDLc and/or HDLc, preferably increasing the levels of circulating HDLc, increasing the HDLc/LDLc ratio, and/or decreasing the levels of circulating LDLc/VLDLc.

Accordingly, the desired therapeutic effect of administering vaccine peptides according to this invention is evidenced by eliciting autoantibodies in an individual that bind endogenous CETP and/or inhibit CETP activity, or by a relative decrease in LDLc and/or VLDLc levels compared to HDLc levels, or by an elevation of absolute levels of circulating HDLc. Because the vaccine peptides of the present invention produce these therapeutically useful effects, the vaccine peptides are useful in the treatment of cardiovascular disease, particularly atherosclerosis. By “treatment” is meant inhibition of progression of a disease or amelioration or reduction of disease symptoms. It is expected that administration of the vaccine peptides of the present invention will prevent or retard the accumulation of atherosclerotic plaque in vaccinated subjects, will arrest the buildup of atherosclerotic plaque, or may even lead to regression of atherosclerotic lesions in treated subjects.

The CETP vaccine peptides of this invention may be administered by any route used for vaccination, including: parenterally such as intraperitoneally, interperitoneally, intradermally, subcutaneously, intramuscularly, or intravenously; or orally. If oral administration of a vaccine peptide is desired, any pharmaceutically acceptable oral excipient may be combined with the vaccine peptides of this invention, such as, for example, the solutions approved for use in the Sabin oral polio vaccine.

Repeat administrations of the vaccine peptides subsequent to the initial priming dose, also known as “booster” administrations, are also contemplated in order to raise or maintain desired circulating anti-CETP antibody titer levels. Biodegradable microspheres, such as those comprised of poly(DL-lactide-co-glycolide), have been shown to be useful for effective vaccine delivery and immunization via oral or parenteral routes (Eldridge et al., in Immunobiology of Proteins and Peptides V: Vaccines: Mechanisms, Design, and Applications, Atassi, M. Z., ed. (Plenum Press, New York, 1989), pp. 191-202)). Because endogenous CETP lacks helper T cell epitopes, endogenous CETP is not expected to boost the autoantibody response elicited by vaccine peptides according to the invention. This differs from traditional vaccines targeting exogenous antigens where re-exposure or challenge by the vaccine target can boost the immune response, for example, leading to elevated antibody titers. For the present invention, it is expected that booster immunizations will be necessary to maintain the autoantibody response capable of inhibiting endogenous CETP.

Appropriate dosages of the peptide vaccines of this invention are established by general vaccine methodologies used in the art based on measurable parameters for which the vaccine is proposed to affect, including monitoring for potential contraindications, such as hypersensitivity reaction, erythema, induration, tenderness (see, e.g., Physicians'Desk Reference, 49^{th} ed., (Medical Economics Data Production Co., Mont Vale, N.J., 1995), pp. 1628, 2371 (referring to hepatitis B vaccine), pp. 1501, 1573, and 1575 (referring to measles, mumps, and/or rubella vaccines), pp. 904, 919, 1247, 1257, 1289, 1293, and 2363 (referring to diptheria, tetanus and/or pertussis vaccines). A common and traditional approach for vaccinating humans is to administer an initial dose of a particular vaccine to sensitize (“prime”) the immune system and then to follow up with one or more “booster” doses of the vaccine to elevate an anamnestic response by the immune system that was sensitized by the initial administration of the vaccine (vaccination). Such a “priming and booster” administration procedure has been well known and commonly used in the art, as for example, in developing and using measles, polio, tetanus, diptheria, and hepatitis B vaccines.

Initially, the amount of a vaccine peptide administered to an individual may be that required to neutralize the approximate level of endogenous CETP activity present in the individual prior to vaccination, as can be determined by measuring CETP activity in serum or plasma samples from the individual, for example as determined using a commercially available CETP assay. Plasma or serum samples from a vaccinated individual can also be monitored to determine whether a measurable increase in the levels of HDL-cholesterol is seen after administration of the vaccine peptide using commercially available assays. A rise in the concentration (titer) of circulating anti-CETP antibodies can be measured in plasma or serum samples, for example using an ELISA assay.

Thus, it is possible and recommended that initially it be established whether a rise in anti-CETP antibody can be correlated with an increase in the level of HDL-cholesterol (i.e., total cholesterol, including cholesteryl esters and unesterified cholesterol, associated with high density lipoprotein), or with a decrease in CETP activity. Thereafter, one needs only to monitor a rise in titer of anti-CETP antibody to determine whether a sufficient dosage of vaccine peptide has been administered or whether a “booster” dose is indicated to elicit an elevated level of anti-CETP antibody. This is the common procedure with various established vaccinations, such a vaccination against hepatitis B virus.

DNA-Based Vaccines

The present invention also contemplates DNA plasmid-based vaccines capable of expressing the autoantigenic peptides of the present invention in situ. Such DNA vaccines are prepared in the form of a plasmid for administration, e.g., by intramuscular injection, to a subject, after which the transcription and translation in vivo of the portion of the plasmid encoding a vaccine peptide according to the present invention leads to production of vaccine peptides that, in turn, elicit the desired autoimmune response described above. A plasmid-based vaccine according to the invention comprises: the structural coding sequence for an autoantigenic fusion polypeptide comprising a DNA sequence encoding at least one universal helper T cell epitope and a DNA sequence encoding at least one B cell epitope from the N-terminus of CETP as described above, which structural coding sequence is operatively linked to a promoter sequence or a promoter/enhancer sequence capable of directing transcription of the structural coding sequence in cells of a mammalian subject. It may also be desirable to include a bacterial origin of replication and a selectable marker(s), for example, to aid in the production of large quantities of the plasmid vaccine in bacterial culture.

A preferred nucleotide sequence encoding a universal helper T cell epitope for use according to the present invention, includes a nucleotide sequence encoding the 14-amino acid tetanus toxin fragment shown in SEQ ID NO:1. A preferred nucleotide sequence encoding this tetanus toxin segment is set forth below: 5′-CAGTACATCAAGGCCAATAGCAAGTTCATCGGCATTACCGAG-3′ (SEQ ID NO: 6).

A preferred nucleotide sequence encoding a CETP B cell epitope for use according to the present invention comprises a nucleotide sequence encoding the N-terminal 21 amino acids of mature human CETP shown in SEQ ID NO:2. A preferred coding sequence for such N-terminal peptide is shown below: 5′-TGTAGCAAGGGCACCTCTCACGAGGCCGGCATCGTGTGCCGGATCACCAAAC CTGCCCTCCTG-3′ (SEQ ID NO: 45). Underlined bases represent silent nucleotide substitutions (i.e., not changing the encoded amino acid) as compared with the native mature human CETP nucleotide sequence (SEQ ID NO:43). The substitutions are optimized for human codon usage.

Another preferred nucleotide sequence encoding a CETP B cell epitope for use in the present invention comprises the nucleotide sequence encoding the CETP N-terminal amino acids 2-21 (SEQ ID NO:3) of human CETP: 5′-AGCAAGGGCACCTCTCACGAGGCCGGCATCGTGTGCCGGATCACCAAACC TGCCCTCCTG-3′ (SEQ ID NO:46). Underlined nucleic acids represent silent nucleotide substitutions as compared with the native CETP nucleotide sequence (SEQ ID NO:43). The substitutions are optimized for human codon usage.

A preferred nucleotide sequence encoding a hybrid peptide for use in a DNA plasmid-based vaccine of the present invention comprises a plasmid insert including the nucleotide sequence encoding the N-terminal 21 amino acids of mature human CETP linked in-frame to the nucleotide sequence encoding a universal helper T cell epitope derived from tetanus toxin: 5′-TGTAGCAAGGGCACCTCTCACGAGGCCGGCATCGTGTGCCGGATCACCAAAC CTGCCCTCCTGCAGTACATCAAGGCCAATAGCAAGTTCATCGGCATTACCGAG-3′ (SEQ ID NO:9). Underlined nucleic acids within the nucleotide sequence encoding the B cell epitope portion represent silent nucleotide substitutions as compared with the native mature human CETP nucleotide sequence (SEQ ID NO:43). The substitutions are optimized for human codon usage.

Another preferred nucleotide sequence for use in a DNA plasmid-based vaccine of the present invention comprises a plasmid insert including a nucleotide sequence encoding amino acids 2-21 from the N-terminus of mature human CETP, linked in-frame to the nucleotide sequence encoding a universal helper T cell epitope derived from tetanus toxin: 5′-AGCAAGGGCACCTCTCACGAGGCCGGCATCGTGTGCCGGATCACCAAACCT GCCCTCCTGCAGTACATCAAGGCCAATAGCAAGTTCATCGGCATTACCGAG-3′ (SEQ ID NO:47). Underlined nucleic acids within the nucleotide sequence encoding the B cell epitope portion represent silent nucleotide substitutions as compared with the native mature human CETP nucleotide sequence (SEQ ID NO:43). The substitutions are optimized for human codon usage.

The DNA plasmid vaccines of the present invention include the nucleotide sequences necessary for in vivo expression of the encoded autoantigenic fusion polypeptide at levels sufficient to elicit production of autoantibodies to the vaccinated subject's endogenous CETP. Transcription of the gene coding for the autoantigenic fusion protein is under the control of a promoter/enhancer sequence. A variety of promoter and enhancer sequences are known in the art and will be suitable for use in the present invention. Preferred promoter/enhancer sequences that may be used in plasmids of this invention include, but are not limited to, the CMV promoter/enhancer sequence, adenovirus promoter/enhancer sequence, and β-actin promoter/enhancer sequence. Whether a particular promoter/enhancer is more or less useful than another promoter/enhancer sequence in the plasmids of this invention can be determined by evaluating the ability of a promoter/enhancer to cause expression of, or to increase the level of expression of a standard reporter gene, such as luciferase or B-galactosidase, and the level of production of antibodies reactive with the expressed reporter in an animal model for gene expression, such as in rabbits or mice.

Generally, the higher the level of expression of the reporter gene product and/or the higher the level of production of antibodies reactive with the expressed reporter gene product, the more useful that particular promoter/enhancer will be at directing transcription of the structural coding sequence for autoantigenic fusion proteins in plasmid-based vaccines according to the invention.

The plasmid-based vaccines according to the invention may be administered in any manner calculated to lead to in vivo expression of the vaccine peptide encoded in the plasmid by the vaccinated subject. Suitable methods of administration include, for instance, direct administration of plasmid DNA via intramuscular injection, intradermal injection or DNA-coated microprojectiles. The amount of vaccine administered will vary widely according to the method of administration, the tissue (for example, skeletal muscle vs. skin) into which the vaccine is administered, the desired titer of anti-CETP antibodies, the particular therapeutic needs of the subject to be immunized, etc. Very large amounts of DNA vaccine, on the order of 10 mg/kg of body weight of the subject, may be administered with injection into muscle tissue, whereas for coated microprojectiles, a much lower dose of the vaccine may be effective for eliciting the desired immune response.

The dosage of vaccine and immunization protocol should be calibrated to obtain a beneficial response, which can be measured in a variety of ways, depending on the clinical setting, for example, by measuring anti-CETP antibody titer, change in lipoprotein profile (for example, increased HDLc level, decreased LDLc level, increased HDLc/LDLc ratio), serum CETP concentration, change (i.e., decrease) in CETP activity, etc. Methods and materials for assessing such titers, lipoprotein levels and CETP activity are well known in the art.

The following examples are provided in order to illustrate the invention described herein. These examples are not intended to in any way limit the scope of the invention.

### EXAMPLES

### Example I

### Initial Screen for Potential CETP B Cell Epitopes

A comprehensive screen for CETP B cell auto-epitopes was performed. Eleven New Zealand dWhite rabbits were immunized each with 0.2 mg of full-length human CETP (huCETP) in Complete Freund's Adjuvant (CFA). The huCETP was purified from conditioned media taken from cultures of a huCETP transformed CHO cell line (obtained from Dr. Alan Tall, Columbia University, New York). The rabbits received two booster immunizations (0.2 mg huCETP in Incomplete Freund's Adjuvant, or IFA), at 5 weeks and 10 weeks after initial vaccination. Serum samples collected 19 weeks after the initial injections were tested for the ability to recognize discrete segments of full-length rabbit CETP (SEQ ID NO:8): the full-length rabbit CETP amino acid sequence was divided into 31 peptides (see Table 1, SEQ ID NOs:10 to 40) comprised of thirty 21-mers and one C-terminal 16-mer, with N-terminal and C-terminal overlaps of five amino acids between adjacent peptides, spanning the entire rabbit CETP sequence. Table 1 (below) shows the amino acid sequence of each of the peptides 1-31 prepared for this experiment (SEQ ID NOs: 10-40).

Sera collected from rabbits immunized with human CETP were analyzed for their ability to recognize, i.e., show antibody binding to, each of the rabbit CETP peptides in the peptide array (Table 1) as a test for cross-reactivity of rabbit anti-human serum to similar rabbit and human CETP peptides. That is, as a test for the elicitation of autoreactive antibodies.

Table 1: Thirty-one overlapping rabbit CETP peptide fragments (cf. SEQ ID NO:8)

TABLE 1

Thirty-one overlapping rabbit CETP peptide
fragments (cf. SEQ ID NO: 8)

| | | | |
|---|---|---|---|
| Peptide | Rabbit CETP | | SEQ ID |
| No. | amino acids | Amino Acid Sequence | NO: |

| | | | |
|---|---|---|---|
| 1 | 1-21 | CPKGASYEAGIVCRITKPALL | 10 |
| 2 | 17-37 | KPALLVLNQETAKVVQTAFQR | 11 |
| 3 | 33-53 | TAFQRAGYPDVSGERAVMLLG | 12 |
| 4 | 49-69 | VMLLGRVKYGLHNLQISHLSI | 13 |
| 5 | 65-85 | SHLSIASSQVELVDAKTIDVA | 14 |
| 6 | 81-101 | TIDVAIQNVSVVFKGTLNYSY | 15 |
| 7 | 97-117 | LNYSYTSAWGLGINQSVDFEI | 16 |
| 8 | 113-133 | VDFEIDSAIDLQINTELTCDA | 17 |
| 9 | 129-149 | LTCDAGSVRTNAPDCYLAFHK | 18 |
| 10 | 145-165 | LAFHKLLLHLQGEREPGWLKQ | 19 |
| 11 | 161-181 | GWLKQLFTNFISFTLKLILKR | 20 |
| 12 | 177-197 | LILKRQVCNEINTISNIMADF | 21 |
| 13 | 193-213 | IMADFVQTRAASILSDGDIGV | 22 |
| 14 | 209-229 | GDIGVDISVTGAPVITATYLE | 23 |
| 15 | 225-245 | ATYLESHHKGHFTHKNVSEAF | 24 |
| 16 | 241-261 | VSEAFPLRAFPPGLLGDSRML | 25 |
| 17 | 257-277 | DSRMLYFWFSDQVLNSLARAA | 26 |
| 18 | 273-293 | LARAAFQEGRLVLSLTGDEFK | 27 |
| 19 | 289-309 | GDEFKKVLETQGFDTNQEIFQ | 28 |
| 20 | 305-325 | QEIFQELSRGLPTGQAQVAVH | 29 |
| 21 | 321-341 | QVAVHCLKVPKISCQNRGVVV | 30 |
| 22 | 337-357 | RGVVVSSSVAVTFRFPRPDGR | 31 |
| 23 | 353-373 | RPDGREAVAYRFEEDIITTVQ | 32 |
| 24 | 369-389 | ITTVQASYSQKKLFLHLLDFQ | 33 |
| 25 | 385-405 | LLDFQCVPASGRAGSSANLSV | 34 |
| 26 | 401-421 | ANLSVALRTEAKAVSNLTESR | 35 |
| 27 | 417-437 | LTESRSESLQSSLRSLIATVG | 36 |
| 28 | 433-453 | IATVGIPEVMSRLEVAFTALM | 37 |
| 29 | 449-469 | FTALMNSKGLDLFEIINPEII | 38 |
| 30 | 465-485 | NPEIITLDGCLLLQMDFGFPK | 39 |
| 31 | 481-496 | FGFPKHLLVDFLQSLS | 40 |

Each of the peptides was linked covalently via its N-terminus to an assigned spot on a modified polypropylene membrane (ResGen, Inc.); the full array of rabbit CETP peptides was presented on a single sheet of membrane. To account for non-specific binding and for binding be pre-existing antibodies, the peptides on the membrane were probed with sera taken from the rabbits one week prior to the prime injection (“pre-bleed” at “week −1” for baseline samples), while another membrane carrying an identical array of peptides was probed with post-vaccination sera. Dot-Blot techniques were employed for the probing of the solid-phase, bound peptides. Secondary antibodies (donkey anti-rabbit Ig) conjugated to horseradish peroxidase (HRP) were used to probe the bound rabbit antibodies, and development was carried out with chemiluminescence reagents (Boehringer Mannheim Corp.).

The results of this analysis are summarized in FIG. 1B. Scoring was done based both on the relative intensity of the signals obtained from week 19 serum samples, and on the presence or absence of immune-recognition in the pre-immune samples. The grading of signals in FIG. 1B is as follows: no signal (−), and weak (±), positive (+), strong (++), or very strong (3+ or 4+) signal. Values are recorded in normal unenclosed type where a signal was detected using only the post-immune (week 19) but not the pre-immune (week −1) sera. Values are recorded in parentheses ( ) where a signal was present both in the pre- and post-immune sera, but the signal was significantly higher in the latter. Values are recorded in brackets [ ] where a signal was observed in both pre- and post-immune sera, but there where no significant differences in their intensities.

The rabbits in FIGS. 1A and 1B are numbered and listed in decreasing order of change in their endogenous serum CETP activity levels, detected at 12 weeks after the first injection with whole human CETP (as compared to week −1 serum CETP activity). At the two extremes are Rabbit #1 with 85% increase in CETP activity following vaccination and Rabbit #11 with 77% decrease in CETP activity following the same treatment (see FIG. 1A). The results in FIG. 1B suggest a correlation between epitope usage and effects of immunization on endogenous CETP activity. In particular, the immune reactivity to peptides 1, 22, 30, and 31 seem to be associated with reduction in CETP activity. Furthermore, reduction in CETP activity appears to be associated with increase in HDL-cholesterol (HDLc). See, FIG. 1A.

Based on these results, four peptides, i.e., peptide 1 (SEQ ID NO:10), peptide 22 (SEQ ID NO:31), peptide 30 (SEQ ID NO:39), and the 16-mer peptide 31 (SEQ ID NO:40), were chosen for further analysis of their potential to function as CETP B cell epitopes in CETP peptide vaccine constructs.

Because our primary interest is in developing CETP vaccines to elicit antibodies recognizing human CETP, the following experiments were carried out using sequences of human CETP that correspond to the rabbit peptides 1, 22, 30, and 31.

### Example II

### Production of Anti-CETP Antibodies in Wild-Type Mice Injected with KLH-Conjugated Peptides

Based on the epitope usage results of Example I, peptides 1 (SEQ ID NO:10), 22 (SEQ ID NO:31), 30 (SEQ ID NO:39), and 31 (SEQ ID NO:40), were selected for further testing for the ability to function as CETP B cell epitopes in a vaccine peptide construct. Human sequences corresponding to those rabbit peptides were determined by comparison of the sequences: FIGS. 8A, 8B and 8C show the respective amino acid sequences of rabbit CETP (SEQ ID NO: 8) and human CETP (SEQ ID NO:7) in alignment. Referring to FIGS. 8A-8C, it is seen that the structure of these two mammalian CETPs is similar, having the same amino acids at 80% of the positions of human CETP. Rabbit CETP (SEQ ID NO:8) is 20 amino acids longer than human CETP (SEQ ID NO:7), and the alignment of the two proteins in FIGS. 8A-8C shows two segments, denoted with dashes (----), where the proteins do not correspond structurally. Positions where the amino acids match between the two proteins are shown by vertical lines (|). Human CETP peptide sequences corresponding to the rabbit peptides used in Example I were determined as set forth in Table 2:

Table 2: Human CETP peptides corresponding to rabbit peptides 1, 22, 30 and 31

TABLE 2

Human CETP peptides corresponding to rabbit
peptides 1, 22, 30 and 31

| | | | |
|---|---|---|---|
| Peptide | CETP | | SEQ ID |
| No. | amino acids | Amino Acid Sequence | NO: |

| | | | |
|---|---|---|---|
| 1 | rabbit 1-21 | CPKGASYEAGIVCRITKPALL | 10 |
| 1 | human 1-21 | CSKGTSHEAGIVCRITKPALL | 51 |
| 22 | rabbit 337-357 | RGVVVSSSVAVTFRFPRPDGR | 31 |
| 22 | human 336-356 | KGVVVNSSVMVKFLFPRPDQQ | 52 |
| 30 | rabbit 465-485 | NPEIITLDGCLLLQMDFGFPK | 39 |
| 30 | human 445-465 | NPEIITRDGFLLLQMDFGFPE | 53 |
| 31 | rabbit 481-496 | FGFPKHLLVDFLQSLS | 40 |
| 31 | human 461-476 | FGFPEHLLVDFLQSLS | 54 |

Referring to FIG. 8A, it is noted that in the N-terminal portion of the human and rabbit CETPs there are only three differences in the respective rabbit and human amino acid sequences, i.e., Pro₂ of the rabbit CETP corresponds to Ser₂ in the human CETP, Ala₅ of the rabbit CETP corresponds to Thr₅ in the human CETP, and Tyr₇ of the rabbit CETP corresponds to His₇ in the human CETP.

Referring to FIGS. 8B and 8C, it is noted that with respect to peptide 22, there are only seven differences in the respective rabbit and human amino acid sequences, i.e., Arg₃₃₇ of the rabbit CETP corresponds to Lys₃₃₆ in the human CETP, Ser₃₄₂ of the rabbit CETP corresponds to Asn₃₄₁ in the human CETP, Ala₃₄₆ of the rabbit CETP corresponds to Met₃₄₅ in the human CETP, Thr₃₄₈ of the rabbit CETP corresponds to Lys₃₄₇ in the human CETP, Arg₃₅₀ of the rabbit CETP corresponds to Leu₃₄₉ in the human CETP, Gly₃₅₆ of the rabbit CETP corresponds to Gln₃₅₅ in the human CETP, and Arg₃₅₇ of the rabbit CETP corresponds to Gln₃₅₆ in the human CETP.

Referring to FIG. 8C, it is noted that with respect to peptide 30, there are only three differences in the respective rabbit and human amino acid sequences, i.e., Leu₄₇₁ of the rabbit CETP corresponds to Arg₄₅₁ in the human CETP, Cys₄₇₄ of the rabbit CETP corresponds to Phe₄₅₄ in the human CETP, and Lys₄₈₅ of the rabbit CETP corresponds to Glu₄₆₅ in the human CETP.

Referring to FIG. 8C, it is noted that in the C-terminal 16-mer (peptide 31) of the human and rabbit CETPs there is only one difference in the respective amino acid sequences, i.e., Lys₄₈₅ of the rabbit CETP corresponds to Glu₄₆₅ in the human CETP.

The human CETP peptides 1, 22, 30, and 31 (Table 2) were conjugated to keyhole limpet hemocyanin (KLH), an immunogenic carrier protein. Peptides 1, 22, and 30 were conjugated through their C-terminus; peptide 31 was conjugated through its N-terminus. The peptide-KLH conjugates were administered to wild-type BALB/c mice with one priming and two booster subcutaneous injections. In the prime immunization each mouse received 0.1 mg of one of the peptides in 1:1 PBS/Complete Freund's Adjuvant (CFA) emulsion. Three weeks later the mice were given a booster injection of 0.1 mg peptide in 1:1 PBS/Incomplete Freund's Adjuvant (IFA) emulsion. Each of the peptide conjugates was used to immunize a group of 5 mice. A fifth group (control) of mice received only KLH (in a PBS/CFA or PBS/IFA emulsion).

FIG. 2 shows the antibody titers to full-length human CETP as determined by ELISA in sera taken 5 weeks after the second booster injection of the human peptide-KLH conjugates (pep-1-KLH, pep-22-KLH, pep-30-KLH, pep-31-KLH in the Figure). In this assay, 100 μl of serially diluted serum samples were added to each well of 96-well plates pre-coated with 0.05 μg recombinant human CETP. The known murine anti-human CETP monoclonal antibody TP2 (obtained from Dr. Alan Tall, Columbia University) was used as a positive control. Immune complexes were probed with sheep anti-mouse IgG conjugated to horseradish peroxidase (HRP). Colorimetric detection was performed with TMB Peroxidase Substrate (Kirkegard & Perry Laboratoreis, Inc. (KPL), Gaithersburg). Peptides 1 and 30 were selected for further study, based on the results illustrated in FIG. 2, which shows that wild-type BALB/c mice immunized with these peptides, conjugated with KLH, generated high levels of human CETP-cross-reactive anti-peptide antibodies, compared with the response to the peptide 22/KLH conjugate or the KLH control. The peptide 31/KLH conjugate, which contained a known CETP B cell epitope (see U.S. Pat. No. 6,410,022), was used in subsequent studies as a control.

### Example III

### Production of Anti-huCETP Antibodies in huCETP Transgenic Mice Injected with KLH-Conjugated Peptides 1, 30, or 31, or a Combination of All Three Peptides

Since mice, unlike humans, do not naturally express endogenous serum CETP, any CETP epitope presented to a wild-type mouse would be foreign to its immune system. Consequently, a wild-type mouse model does not mimic the conditions in a human patient where a CETP vaccine would be directed at an auto-antigen. To test the CETP vaccines in a mammalian system that would require overcoming the endogenous immune recognition of “self”, the subsequent experiments were carried out using transgenic mice expressing human CETP (Taconic, Germantown, N.Y.).

Human CETP transgenic mice were immunized by subcutaneous injection with KLH-conjugated human CETP peptides 1, 30, and 31, using the same protocol employed in Example II. The conjugates were injected (0.1 mg peptide in 0.2 ml PBS/CFA emulsion per rabbit per administration) into three groups of mice (one peptide conjugate/group; 9-10 mice/group); a fourth group received a mixture of all three peptide conjugates (0.1 mg of each peptide in 0.2 ml total of PBS/CFA emulsion per rabbit per administration) to test for potential additive responses. A booster vaccination (in IFA) was given 10 weeks later. A fifth group of mice served as negative controls, receiving PBS/CFA or PBS/IFA emulsion only. Serum samples were collected at 10 and 13 weeks post immunization and anti-CETP titers determined as described in Example II. The results are shown in FIG. 3.

As seen in FIG. 3, the level of response to peptide-1-KLH was considerably higher in the transgenic model than in the wild-type mouse model (FIG. 2). In both models, 100% of the mice responded to the peptide 1 (CETP N-terminal region) conjugate. Peptide-30-KLH raised an antibody titer in only one out of nine transgenic mice, resembling results obtained previously with the non-transgenic mice. No transgenic mouse showed an immune response to peptide-31-KLH, however this is believed to be a result of human error since an earlier experiment with the same peptide and mouse strain resulted in high anti-CETP titers.

In the group receiving a mixture of all three peptide conjugates, eight out of nine mice were positive for anti-huCETP titers. These titers were somewhat lower than when immunized with peptide-1-KLH alone, suggesting no additive immune responses and, moreover, implying interference in the immune reactivity to the epitopes presented by these peptides.

### Example IV

### Changes in CETP Activity in huCETP Transgenic Mice Following Injection of KLH-Conjugated Peptides 1, 30, or 31

Sera collected in the Example III experiment was used to test effects of stimulating anti-CETP immune response with KLH-conjugated peptide vaccines on serum CETP activity. The method used to determine serum CETP activity was based on the assay described in C. Bisgaier et al., J. Lipid Res., 34:1625 (1993). CETP activity in a sample is measured as the degree of fluorescence de-quenching that results from CETP-mediated transfer of fluorescent-labeled cholesteryl ester from Donor to Accepter synthetic lipid micro-emulsions, which are added to the reaction mix at the onset of the reaction period. The synthetic Donor micro-emulsions are composed of 1-palmitoyl-2-oleoyl-phosphatidylcholine (POPC, Avanti Polar-Lipids, Inc., Alabaster, Ala.), triolein, cholesteryl oleate and BODIPY-FL-cholesteryl (a fluorescent-labeled cholesteryl ester; Molecular Probes, Inc., Eugene, Oreg.). BODIPY-FL-cholesteryl is not included in the Acceptor micro-emulsions preparation, which are otherwise identical to that of Donor micro-emulsions. A mixture consisting of the test sample (whole serum), and Donor and Acceptor micro-emulsions, is incubated in a well of a black 96-well plate at 37° C. for 20 hours. At the end of the incubation period, a reading is taken with the aid of a fluorescence plate reader (CytoFluor™ II Microplate, Millipore, Inc., Billerica, Mass.; 485 nm excitation filter; 530 nm emission filter).

The results of the CETP activity analysis is shown in FIGS. 4A and 4B. FIG. 4A shows the average change in CETP activity in sera from huCETP transgenic mice that received two vaccine administrations (prime and one booster), collected 13 weeks after prime immunizations. FIG. 4B shows the average change in CETP in sera from the same mice, collected at 19 weeks post prime immunizations (and 6 weeks after a second booster injection). The data are presented as a percent change in CETP activity in the post-immune sera relative to that found in the pre-immune sera for each mouse. Control mice injected with KLH only showed a marked increase in their serum CETP activity (150-450%) in the “post-immune” sera. The reason for this increase is not clear but may be a natural aging phenomenon in huCETP transgenic mice, as this increase has been observed in other experiments with this animal model (data not shown).

FIG. 4A (sera taken at 5 weeks after the first booster injection), shows that the vaccination of human CETP transgenic mice with either the human CETP peptide 1 conjugate (pep-1-KLH) or the peptide 30 conjugate (pep-30-KLH) resulted in approximately 67% and 70% reduction in CETP activity, respectively, as compared with the CETP activity in control mice injected with KLH alone. This reduction is grater than that obvserved with the peptide 31 conjugate (pep-31-KLH), which led to a reduction of CETP activity of 56%. Vaccination with a combination of the three conjugates, pep-1-KLH, pep-30-KLH and pep-31-KLH, led to a reduction in CETP activity compared to KLH control of about 82%, indicating that the effect of compound immunizations can be additive. This was unexpected, as this group of mice had a lower antibody titer to CETP than the groups immunized with the individual peptides (see Example III).

FIG. 4B illustrates the changes in levels of serum CETP activity in human CETP transgenic mice after receiving the second booster injection of KLH-conjugated peptides 1, 30, or 31. KLH injected alone was used as a control. Data are presented as a percent change in CETP activity detected post-immunization relative to the pre-immune sera. As seen in FIG. 4B, vaccination with pep-1-KLH reduced CETP activity by approximately 65% relative to mice injected with KLH alone, while vaccination with pep-30-KLH and pep-31-KLH led to reduction in CETP activity of about 52% and 22%, respectively, compared to transgenic mice injected with KLH alone.

### Example V

### Changes in Total CETP Activity and Levels of HDLc in huCETP Transgenic Mice Injected with CETP Vaccine Peptides

Human CETP transgenic mice were immunized by subcutaneous injection with a CETP vaccine peptide according to the invention composed of human CETP peptide 1 (SEQ ID NO:51) covalently linked to a 14-mer universal (broad range) helper T cell epitope derived from tetanus toxin, QYIKANSKFIGITE (SEQ ID NO:1). To minimize masking of the functional epitope(s) in the CETP peptide, the tetanus toxin 14-mer was linked via its N-terminus to peptide 1, giving the vaccine peptide having the sequence: CSKGTSHEAGIVCRITKPALLQYIKANSKFIGITE (SEQ ID NO:4). This peptide was designated CETi-2.

For comparison, a positive control vaccine peptide was synthesized utilizing the C-terminal CETP peptide (i.e., human CETP peptide 31, SEQ ID NO:54) linked to the same tetanus toxin universal helper T cell epitope (here through the C-terminus of the tetanus toxin-derived peptide). This peptide was designated CETi-1 and had the following amino acid sequence: CQYIKANSKFIGITEFGFPEHLLVDFLQSLS (SEQ ID NO:41). The CETi-1 vaccine peptide was identical to vaccine peptide CETi-1 described in U.S. Pat. No. 6,410,022. This peptide carries an added (non-native) amino-terminal cysteine to aid in dimerization.

A further vaccine peptide according to the invention was synthesized which was identical CETi-2 described above, except that it lacked the native N-terminal cysteine residue. The N-terminal region of human CETP contains two cysteine residues (at positions 1 and 13) that can facilitate the formation of intra-molecular and inter-molecular disulfide bonds, and thus lead to the unintended creation of complex structures. We found that in the absence of the first cysteine, the peptide's tertiary structural complexity in solution is reduced significantly (data not shown). This peptide, comprising human CETP amino acids 2-21 linked to a C-terminal universal helper T cell epitope (SEQ ID NO:1) had the amino acid sequence: SKGTSHEAGIVCRITKPALLQYIKANSKFIGITE (SEQ ID NO:5) and was designated CETi-2.1.

Each of the three peptides CETi-1, CETi-2, and CETi-2.1 were used to immunize a group of 8-10 huCETP transgenic mice. A fourth group of mice was immunized with a mixture of CETi-1 and CETi-2. In the priming vaccination, each mouse received 0.1 mg of vaccine peptide in PBS/CFA emulsion; while in the two subsequent booster vaccinations (5 and 10 weeks later), IFA was substituted for CFA. A fifth group, serving as a negative control, received PBS/CFA emulsion or PBS/IFA emulsion injections only. All injections were subcutaneous injections at the base of the tail.

Serum samples were collected for evaluation three weeks after the third injection (second booster vaccination). FIG. 5 shows anti-huCETP titers of individual mice and the average values (represented by bars) determined for each group of mice. Similar average levels of immune response were obtained with CETi-2 and CETi-1 (OD˜0.750 at a 1:1000 dilution) vaccinations. The titers of the groups receiving CETi-2.1 alone, or a mixture of CETi-1 and CETi-2, were lower than those of the previous two groups (by approx. 30%).

Serum CETP activity levels for each of the mice and their average values (bars) are represented in FIG. 6. As in Example IV, untreated control mice showed an approximately 300% increase in CETP activity over the course of the experiment. CETi-2 showed no effect on average, while CETi-1 appeared to elevate CETP activity (instead of decreasing it as expected, and as seen in Example IV with the peptide-1-KLH conjugate). In contrast, reduction in CETP activity (by 15%) was observed in the group that received CETi-1 and CETi-2 together, and dramatic reduction in CETP activity (by ˜60%) was observed in the group vaccinated with CETi-2.1 alone.

Finally, FIG. 7 shows results of serum HDL-cholesterol (HDLc) analysis. Baseline (no treatment control group) HDLc levels appeared to increase over the course of the experiment and may be a natural phenomenon of aging in these transgenic animals. In contrast to the control group, the mice that received CETi-1, CETi-2, or a mixture of both CETi-1 and CETi-2 showed reduction in their serum HDLC levels (most pronounced in the group receiving both vaccine peptides). Conversely, a significant increase (by 48% relative to control) in serum HDLc levels was seen with the group of mice that received CETi-2.1 vaccine peptide. These results correlate well with the CETP activity results obtained with the same samples (FIG. 6), suggesting that the vaccination-induced reduction in CETP activity led to the desired outcome, that is increase in HDLc, or “good cholesterol”.

These results demonstrate the lack of a tight correlation between the levels of antibody titers raised by the anti-CETP vaccines peptides and their effectiveness in controlling serum CETP activity or HDLc levels. Thus, the site of interaction of the induced auto-antibodies with endogenous CETP may be more important. The very different effects obtained with CETi-2 and CETi-2.1 are most likely due to the differences in their secondary and tertiary structures, as their primary structures are practically identical. CETi-2, which has two Cys residues, at positions 1 and 13 of the peptide, can spontaneously encycle or polymerize by oxidation to form complex structures; whereas CETi-2.1, which is missing the Cys residue at position 1, would at most form vaccine peptide dimers when oxidized.

From the foregoing description it is evident that an autoantigenic vaccine peptide utilizing the N-terminal portion of cholesteryl ester transfer protein provides an unexpectedly effective autoimmunogen for eliciting an antibody response in a vaccinated individual that concomitantly controls (decreases) serum CETP activity and increases serum HDLc level.

Although a number of embodiments have been described above, it will be understood by those skilled in the art that modifications and variations of the described compositions and methods may be made without departing from the disclosure of the invention or the scope of the appended claims. The articles and publications cited above are hereby incorporated by reference.

Index of SEQ ID NOs.

| SEQ ID | |
|---|---|
| NO: | Description |
| 1 | 14-amino acid sequence of a universal helper T cell epitope from |
| | tetanus toxin |
| 2 | the 21 N-terminal amino acids of human CETP |
| 3 | amino acids 2-21 of human CETP |
| 4 | autoantigenic polypeptide according to the present invention |
| | (CETi-2) |
| 5 | autoantigenic polypeptide according to the present invention |
| | (CETi-2.1) |
| 6 | nucleotide sequence encoding a 14-amino acid universal helper |
| | T cell epitope, a peptide fragment from tetanus toxin |
| 7 | amino acid sequence of full-length mature human CETP |
| 8 | amino acid sequence of full-length mature rabbit CETP |
| 9 | nucleotide sequence encoding CETi-2 vaccine peptide |
| 10-40 | amino acid sequences of 30 overlapping 21-mer rabbit CETP |
| | peptides and C-terminal 16-amino acid peptide (see Table 1) |
| 41 | amino acid sequence of polypeptide designated CETi-1 |
| 42 | a pan-DR universal helper T cell epitope peptide |
| 43 | nucleotide sequence coding for mature human CETP |
| 44 | nucleotide sequence coding for mature rabbit CETP |
| 45 | nucleotide sequence encoding N-terminal 21 amino acids of |
| | human CETP |
| 46 | nucleotide sequence encoding amino acids 2-21 of human CETP |
| 47 | nucleotide sequence encoding CETi-2.1 vaccine peptide |
| 48 | a pan-DR universal helper T cell epitope peptide |
| 49 | a pan-DR universal helper T cell epitope peptide |
| 50 | 21-amino acid sequence of a universal helper T cell epitope, |
| | a peptide fragment of tetanus toxin |
| 51-54 | amino acid sequences of four human CETP peptides |
| | corresponding to rabbit CETP peptides 1, 22, 30 and 31 |
| | (shown in Table 2, supra) |

What we claim is:

1. An isolated autoantigenic hybrid peptide comprising a universal helper T cell epitope portion linked to a cholesteryl ester transfer protein (CETP) B cell epitope portion, wherein said B cell epitope portion comprises from 6 to 21 consecutive amino acids of the amino-terminal 21 amino acids of cholesteryl ester transfer protein.

2. The autoantigenic hybrid peptide according to claim 1, wherein said B cell epitope portion is comprised of from 6 to 21 consecutive amino acids of the amino-terminal 21 amino acids (SEQ ID NO:2) of human cholesteryl ester transfer protein (SEQ ID NO:7).

3. The autoantigenic hybrid peptide of claim 2, wherein the B cell epitope portion of said hybrid peptide is selected from the group consisting of the amino acid sequence of SEQ ID NO:2 and the amino acid sequence of SEQ ID NO:3.

4. The autoantigenic hybrid peptide according to claim 2, wherein the universal helper T cell epitope is selected from the group consisting of a universal helper T cell epitope amino acid sequence of tetanus toxin, diphtheria toxin, pertussis vaccine, Bacile Calmette-Guerin (BCG), polio vaccine, measles vaccine, mumps vaccine, rubella vaccine, purified protein derivative of tuberculin, keyhole limpet hemocyanin, heat shock protein HSP65 or HSP70 from M. tuberculosis, synthetic pan-DR epitope peptides, and combinations or repeats thereof.

5. The autoantigenic hybrid peptide according to claim 2, wherein the universal helper T cell epitope portion comprises a member of the group consisting of QYIKANSKFIGITE (SEQ ID NO: 1); FNNFTVSFWLRVP KVSASHLE (SEQ ID NO:50); X₁KX₂VAAWTLKAX₁ (SEQ ID NO:42), X₁KX₂VAAWTLKAAX₁ (SEQ ID NO:48), or AKX₂VAAWTLKAAA (SEQ ID NO:49), wherein X₁ is D-Ala and X₂ is cyclohexylalanine; combinations thereof; and repeats thereof.

6. An autoantigenic hybrid peptide comprising the amino acid sequence of SEQ ID NO:4.

7. An autoantigenic hybrid peptide comprising the amino acid sequence of SEQ ID NO:5.

8. The autoantigenic hybrid peptide according to claim 6 consisting of the amino acid sequence of SEQ ID NO:4.

9. The autoantigenic hybrid peptide according to claim 7 consisting of the amino acid sequence of SEQ ID NO:5.

10. The autoantigenic hybrid peptide according to claim 6, wherein said hybrid peptide is a monomer, dimer, trimer, or tetramer of SEQ ID NO:4, or a mixture of thereof.

11. The autoantigenic hybrid peptide according to claim 7, wherein said hybrid peptide is a monomer, dimer, trimer, or tetramer of SEQ ID NO:5, or a mixture thereof.

12. A pharmaceutical composition comprising an autoantigenic hybrid peptide according to any one of claims 1-11, or a mixture of such peptides, dispersed in a pharmaceutically acceptable carrier.

13. A method of elevating the ratio of circulating HDLc to circulating LDLc, VLDLc, or total cholesterol in a mammalian subject comprising administering to a mammal an autoantigenic hybrid peptide comprising a universal helper T cell epitope portion and a CETP B cell epitope portion, wherein said B cell epitope portion comprises from 6 to 21 consecutive amino acids of the amino-terminal 21 amino acids of cholesteryl ester transfer protein (CETP).

14. The method according to claim 13, wherein the B cell epitope portion comprises from 6 to 21 consecutive amino acids of the amino-terminal 21 amino acids corresponding to said mammal's endogenous CETP.

15. The method according to claim 13, wherein the universal helper T cell epitope is selected from the group consisting of a universal helper T cell epitope amino acid sequence of tetanus toxin, diphtheria toxin, pertussis vaccine, Bacile Calmette-Guerin (BCG), polio vaccine, measles vaccine, mumps vaccine, rubella vaccine, purified protein derivative of tuberculin, keyhole limpet hemocyanin, heat shock protein HSP65 or HSP70 from M. tuberculosis, synthetic pan-DR epitope peptides, and combinations or repeats thereof.

16. The method according to claim 13, wherein the universal helper T cell epitope portion comprises a member of the group consisting of QYIKANSKFIGITE (SEQ ID NO: 1); FNNFTVSFWLRVP KVSASHLE (SEQ ID NO:50); X₁KX₂VAAWTLKAX₁ (SEQ ID NO:42), X₁KX₂VAAWTLKAAX, (SEQ ID NO:48), or AKX₂VAAWTLKAAA (SEQ ID NO:49), wherein X₁ is D-Ala and X₂ is cyclohexylalanine; combinations thereof; and repeats thereof.

17. The method according to claim 13, wherein the universal helper T cell epitope portion of the hybrid peptide is selected from the group consisting of amino acids 830 to 843 of tetanus toxin protein (SEQ ID NO:1) or the amino acid sequence of amino acids 947 to 967 of tetanus toxin protein (SEQ ID NO:50).

18. The method according to claim 13, wherein said hybrid peptide comprises the sequence of amino acids of SEQ ID NO:4.

19. The method according to claim 13, wherein said hybrid peptide comprises the sequence of amino acids of SEQ ID NO:5.

20. The method according to claim 18, wherein said hybrid peptide consists of the sequence of amino acids of SEQ ID NO:4.

21. The method according to claim 19, wherein said hybrid peptide consists of the sequence of amino acids of SEQ ID NO:5.

22. The method according to claim 18, wherein said autoantigenic hybrid peptide is a monomer, dimer, trimer, or tetramer of SEQ ID NO:4, or a mixture thereof.

23. The method according to claim 19, wherein said autoantigenic hybrid peptide is a monomer, dimer, trimer, or tetramer of SEQ ID NO:5, or a mixture thereof.

24. The method according to claim 13, comprising the further step of repeating said administering step one or more times.

25. The method according to claim 13, wherein said autoantigenic hybrid peptide is administered in combination with one or more different autoantigenic hybrid peptides.

26. The method according to claim 25, wherein said one or more different autoantigenic hybrid peptides includes a peptide having the sequence of SEQ ID NO:41.

27. A method of decreasing the level of CETP activity in a mammalian subject comprising administering to a mammal an autoantigenic hybrid peptide comprising a universal helper T cell epitope portion linked to a CETP B cell epitope portion, wherein said B cell epitope portion comprises from 6 to 21 consecutive amino acids of the amino-terminal 21 amino acids of cholesteryl ester transfer protein (CETP).

28. The method according to claim 27, wherein the B cell epitope portion comprises from 6 to 21 consecutive amino acids of the amino-terminal 21 amino acids corresponding to said mammal's endogenous CETP.

29. The method according to claim 27, wherein the universal helper T cell epitope is selected from the group consisting of a universal helper T cell epitope amino acid sequence of tetanus toxin, diphtheria toxin, pertussis vaccine, Bacile Calmette-Guerin (BCG), polio vaccine, measles vaccine, mumps vaccine, rubella vaccine, purified protein derivative of tuberculin, keyhole limpet hemocyanin, heat shock protein HSP65 or HSP70 from M. tuberculosis, synthetic pan-DR epitope peptides, and combinations or repeats thereof.

30. The method according to claim 27, wherein the universal helper T cell epitope portion comprises a member of the group consisting of QYIKANSKFIGITE (SEQ ID NO: 1); FNNFTVSFWLRVP KVSASHLE (SEQ ID NO:50); X₁KX₂VAAWTLKAX₁ (SEQ ID NO:42), X₁KX₂VAAWTLKAAX₁ (SEQ ID NO:48), or AKX₂VAAWTLKAAA (SEQ ID NO:49), wherein X₁ is D-Ala and X₂ is cyclohexylalanine; combinations thereof; and repeats thereof.

31. The method according to claim 27, wherein the universal helper T cell epitope portion of the hybrid peptide is selected from the group consisting of amino acids 830 to 843 of tetanus toxin protein (SEQ ID NO:1) or the amino acid sequence of amino acids 947 to 967 of tetanus toxin protein (SEQ ID NO:50).

32. The method according to claim 27, wherein said hybrid peptide comprises the sequence of amino acids of SEQ ID NO:4.

33. The method according to claim 27, wherein said hybrid peptide comprises the sequence of amino acids of SEQ ID NO:5.

34. The method according to claim 32, wherein said hybrid peptide consists of the sequence of amino acids of SEQ ID NO:4.

35. The method according to claim 33, wherein said hybrid peptide consists of the sequence of amino acids of SEQ ID NO:5.

36. The method according to claim 32, wherein said autoantigenic hybrid peptide is a monomer, dimer, trimer, or tetramer of SEQ ID NO:4, or a mixture thereof.

37. The method according to claim 33, wherein said autoantigenic hybrid peptide is a monomer, dimer, trimer, or tetramer of SEQ ID NO:5, or a mixture thereof.

38. The method according to claim 27, comprising the further step of repeating said administering step one or more times.

39. The method according to claim 27, wherein said autoantigenic hybrid peptide is administered in combination with one or more different autoantigenic hybrid peptides.

40. The method according to claim 39, wherein said one or more different autoantigenic hybrid peptides includes a peptide having the sequence of SEQ ID NO:41.

41. A method of increasing the level of circulating HDLc in a mammalian subject comprising administering to the mammal an autoantigenic hybrid peptide comprising a universal helper T cell epitope portion and a CETP B cell epitope portion, wherein said B cell epitope portion comprises from 6 to 21 consecutive amino acids of the amino-terminal 21 amino acids of cholesteryl ester transfer protein (CETP).

42. The method according to claim 41, wherein the B cell epitope portion comprises from 6 to 21 consecutive amino acids of the amino-terminal 21 amino acids corresponding to said mammal's endogenous CETP.

42. The method according to claim 41, wherein the universal helper T cell epitope is selected from the group consisting of a universal helper T cell epitope amino acid sequence of tetanus toxin, diphtheria toxin, pertussis vaccine, Bacile Calmette-Guerin (BCG), polio vaccine, measles vaccine, mumps vaccine, rubella vaccine, purified protein derivative of tuberculin, keyhole limpet hemocyanin, heat shock protein HSP65 or HSP70 from M. tuberculosis, synthetic pan-DR epitope peptides, and combinations or repeats thereof.

43. The method according to claim 41, wherein the universal helper T cell epitope portion comprises a member of the group consisting of QYIKANSKFIGITE (SEQ ID NO: 1); FNNFTVSFWLRVP KVSASHLE (SEQ ID NO:50); X₁KX₂VAAWTLKAX₁ (SEQ ID NO:42), X₁KX₂VAAWTLKAAX₁ (SEQ ID NO:48), or AKX₂VAAWTLKAAA (SEQ ID NO:49), wherein X₁ is D-Ala and X₂ is cyclohexylalanine; combinations thereof; and repeats thereof.

44. The method according to claim 41, wherein the universal helper T cell epitope portion of the hybrid peptide is selected from the group consisting of amino acids 830 to 843 of tetanus toxin protein (SEQ ID NO:1) or the amino acid sequence of amino acids 947 to 967 of tetanus toxin protein (SEQ ID NO:50).

45. The method according to claim 41, wherein said hybrid peptide comprises the sequence of amino acids of SEQ ID NO:4.

46. The method according to claim 41, wherein said hybrid peptide comprises the sequence of amino acids of SEQ ID NO:5.

47. The method according to claim 45, wherein said hybrid peptide consists of the sequence of amino acids of SEQ ID NO:4.

48. The method according to claim 46, wherein said hybrid peptide consists of the sequence of amino acids of SEQ ID NO:5.

49. The method according to claim 45, wherein said autoantigenic hybrid peptide is a monomer, dimer, trimer, or tetramer of SEQ ID NO:4, or a mixture thereof.

50. The method according to claim 47, wherein said autoantigenic hybrid peptide is a monomer, dimer, trimer, or tetramer of SEQ ID NO:5, or a mixture thereof.

51. The method according to claim 41, comprising the further step of repeating said administering step one or more times.

52. The method according to claim 41, wherein said autoantigenic hybrid peptide is administered in combination with one or more different autoantigenic hybrid peptides.

53. The method according to claim 52, wherein said one or more different autoantigenic hybrid peptides includes a peptide having the sequence of SEQ ID NO:41.

54. A method of making an anti-cholesteryl ester transfer protein (CETP) vaccine peptide comprising:
a) selecting a B cell epitope portion comprising from 6 to 21 consecutive amino acids of the N-terminal 21 amino acids of a CETP;
b) selecting a universal helper T cell epitope portion comprising a universal helper T cell epitope; and
c) linking said B cell epitope portion and said universal helper T cell epitope portion to form a single autoantigenic moiety.

55. The method according to claim 54, wherein said B cell epitope portion is covalently linked to said universal helper T cell epitope portion.

56. The method according to claim 55, wherein said B cell epitope portion is covalently linked to said universal helper T cell epitope portion via a covalent bond selected from the group consisting of peptide bonds and disulfide bonds.

57. The method according to claim 54, wherein said B cell epitope portion is linked to said universal helper T cell epitope portion via a cross-linker molecule.

58. The method according to claim 54, wherein said B cell epitope portion is linked to said universal helper T cell epitope portion via a bridge of amino acids.

59. The method according to claim 54, wherein said B cell epitope portion and said universal helper T cell epitope portion are linked to a common carrier molecule.

60. The method according to claim 54, wherein said B cell epitope portion is linked to said universal helper T cell epitope portion to form a vaccine peptide and further comprising the step of linking said vaccine peptide to a carrier molecule.

61. A plasmid-based autoantigenic composition comprising a polynucleotide comprising a nucleotide sequence coding for an autoantigenic hybrid peptide, which nucleotide sequence includes at least one segment coding for 6 to 21 consecutive amino acids of the amino-terminal 21 amino acids of a cholesteryl ester transfer protein (CETP) linked in-frame with at least one segment coding for a universal helper T cell epitope, which nucleotide sequence is operably linked to a promoter sequence suitable for directing the transcription of the nucleotide sequence in a mammalian cell.

62. The plasmid-based autoantigenic composition according to claim 61, wherein said CETP is human CETP and said promoter sequence is suitable for directing transcription of the nucleotide sequence in a human cell.

63. The plasmid-based autoantigenic composition according to claim 62, wherein said amino-terminal 21 amino acids has the sequence of SEQ ID NO:2.

64. The plasmid-based autoantigenic composition according to claim 63, wherein the universal helper T cell epitope is selected from the group consisting of a universal helper T cell epitope amino acid sequence of tetanus toxin, diphtheria toxin, pertussis vaccine, Bacile Calmette-Guerin (BCG), polio vaccine, measles vaccine, mumps vaccine, rubella vaccine, purified protein derivative of tuberculin, keyhole limpet hemocyanin, heat shock protein HSP65 or HSP70 from M. tuberculosis, synthetic pan-DR epitope peptides, and combinations or repeats thereof.

65. The plasmid-based autoantigenic composition according to claim 61, wherein said nucleotide sequence encodes a polypeptide having the sequence of SEQ ID NO:4 or SEQ ID NO:5.


SEQUENCE LISTING (54 sequences)

SEQ ID NO: 1 - protein, 14 aa, Clostridium tetani
  PEPTIDE (1)..(14): 14 amino acid sequence of a universal helper T cell epitope from tetanus toxoid (tt)
QYIKANSKFIGITE

SEQ ID NO: 2 - protein, 21 aa, Human
  PEPTIDE (1)..(21): 21-amino terminal amino acids of human cholesteryl ester transfer protein (CETP)
CSKGTSHEAGIVCRITKPALL

SEQ ID NO: 3 - protein, 20 aa, Human
  PEPTIDE (1)..(20): amino acids 2-21 of human CETP
SKGTSHEAGIVCRITKPALL

SEQ ID NO: 4 - protein, 35 aa, Artificial
  synthetic hybrid peptide
CSKGTSHEAGIVCRITKPALLQYIKANSKFIGITE

SEQ ID NO: 5 - protein, 34 aa, Artificial
  synthetic hybrid peptide
SKGTSHEAGIVCRITKPALLQYIKANSKFIGITE

SEQ ID NO: 6 - DNA, 42 bp, Clostridium tetani
  misc_feature (1)..(42): nucleotide sequence encoding a 14-amino acid tetanus toxoid universal helper T cell epitope
cagtacatcaaggccaatagcaagttcatcggcattaccgag

SEQ ID NO: 7 - protein, 476 aa, Human
  PEPTIDE (1)..(476): Amino aid sequence of full-length mature human CETP protein
CSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYTFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 8 - protein, 496 aa, Rabbit
  PEPTIDE (1)..(496): Amino acid sequence of full-length mature rabbit CETP protein
CPKGASYEAGIVCRITKPALLVLNQETAKVVQTAFQRAGYPDVSGERAVMLLGRVKYGLHNLQISHLSIASSQVELVDAKTIDVAIQNVSVVFKGTLNYSYTSAWGLGINQSVDFEIDSAIDLQINTELTCDAGSVRTNAPDCYLAFHKLLLHLQGEREPGWLKQLFTNFISFTLKLILKRQVCNEINTISNIMADFVQTRAASILSDGDIGVDISVTGAPVITATYLESHHKGHFTHKNVSEAFPLRAFPPGLLGDSRMLYFWFSDQVLNSLARAAFQEGRLVLSLTGDEFKKVLETQGFDTNQEIFQELSRGLPTGQAQVAVHCLKVPKISCQNRGVVVSSSVAVTFRFPRPDGREAVAYRFEEDIITTVQASYSQKKLFLHLLDFQCVPASGRAGSSANLSVALRTEAKAVSNLTESRSESLQSSLRSLIATVGIPEVMSRLEVAFTALMNSKGLDLFEIINPEIITLDGCLLLQMDFGFPKHLLVDFLQSLS

SEQ ID NO: 9 - DNA, 105 bp, Artificial
  synthetic hybrid sequence
tgtagcaagggcacctctcacgaggccggcatcgtgtgccggatcaccaaacctgccctcctgcagtacatcaaggccaatagcaagttcatcggcattaccgag

SEQ ID NO: 10 - protein, 21 aa, Rabbit
  FRAGMENT OF PEPTIDE (1)..(21): fragment of rabbit CETP peptide
CPKGASYEAGIVCRITKPALL

SEQ ID NO: 11 - protein, 21 aa, Rabbit
  FRAGMENT OF PEPTIDE (1)..(21): fragment of rabbit CETP peptide
KPALLVLNQETAKVVQTAFQR

SEQ ID NO: 12 - protein, 21 aa, Rabbit
  FRAGMENT OF PEPTIDE (1)..(21): fragment of rabbit CETP peptide
TAFQRAGYPDVSGERAVMLLG

SEQ ID NO: 13 - protein, 21 aa, Rabbit
  FRAGMENT OF PEPTIDE (1)..(21): fragment of rabbit CETP peptide
VMLLGRVKYGLHNLQISHLSI

SEQ ID NO: 14 - protein, 21 aa, Rabbit
  FRAGMENT OF PEPTIDE (1)..(21): fragment of rabbit CETP peptide
SHLSIASSQVELVDAKTIDVA

SEQ ID NO: 15 - protein, 21 aa, Rabbit
  FRAGMENT OF PEPTIDE (1)..(21): fragment of rabbit CETP peptide
TIDVAIQNVSVVFKGTLNYSY

SEQ ID NO: 16 - protein, 21 aa, Rabbit
  FRAGMENT OF PEPTIDE (1)..(21): fragment of rabbit CETP peptide
LNYSYTSAWGLGINQSVDFEI

SEQ ID NO: 17 - protein, 21 aa, Rabbit
  FRAGMENT OF PEPTIDE (1)..(21): fragment of rabbit CETP peptide
VDFEIDSAIDLQINTELTCDA

SEQ ID NO: 18 - protein, 21 aa, Rabbit
  FRAGMENT OF PEPTIDE (1)..(21): fragment of rabbit CETP peptide
LTCDAGSVRTNAPDCYLAFHK

SEQ ID NO: 19 - protein, 21 aa, Rabbit
  FRAGMENT OF PEPTIDE (1)..(21): fragment of rabbit CETP peptide
LAFHKLLLHLQGEREPGWLKQ

SEQ ID NO: 20 - protein, 21 aa, Rabbit
  FRAGMENT OF PEPTIDE (1)..(21): fragment of rabbit CETP peptide
GWLKQLFTNFISFTLKLILKR

SEQ ID NO: 21 - protein, 21 aa, Rabbit
  FRAGMENT OF PEPTIDE (1)..(21): fragment of rabbit CETP peptide
LILKRQVCNEINTISNIMADF

SEQ ID NO: 22 - protein, 21 aa, Rabbit
  FRAGMENT OF PEPTIDE (1)..(21): fragment of rabbit CETP peptide
IMADFVQTRAASILSDGDIGV

SEQ ID NO: 23 - protein, 21 aa, Rabbit
  FRAGMENT OF PEPTIDE (1)..(21): fragment of rabbit CETP peptide
GDIGVDISVTGAPVITATYLE

SEQ ID NO: 24 - protein, 21 aa, Rabbit
  FRAGMENT OF PEPTIDE (1)..(21): fragment of rabbit CETP peptide
ATYLESHHKGHFTHKNVSEAF

SEQ ID NO: 25 - protein, 21 aa, Rabbit
  FRAGMENT OF PEPTIDE (1)..(21): fragment of rabbit CETP peptde
VSEAFPLRAFPPGLLGDSRML

SEQ ID NO: 26 - protein, 21 aa, Rabbit
  FRAGMENT OF PEPTIDE (1)..(21): fragment of rabbit CETP peptide
DSRMLYFWFSDQVLNSLARAA

SEQ ID NO: 27 - protein, 21 aa, Rabbit
  FRAGMENT OF PEPTIDE (1)..(21): fragment of rabbit CETP peptide
LARAAFQEGRLVLSLTGDEFK

SEQ ID NO: 28 - protein, 21 aa, Rabbit
  FRAGMENT OF PEPTIDE (1)..(21): fragment of rabbit CETP peptide
GDEFKKVLETQGFDTNQEIFQ

SEQ ID NO: 29 - protein, 21 aa, Rabbit
  FRAGMENT OF PEPTIDE (1)..(21): fragment of rabbit CETP peptide
QEIFQELSRGLPTGQAQVAVH

SEQ ID NO: 30 - protein, 21 aa, Rabbit
  FRAGMENT OF PEPTIDE (1)..(21): fragment of rabbit CETP peptide
QVAVHCLKVPKISCQNRGVVV

SEQ ID NO: 31 - protein, 21 aa, Rabbit
  FRAGMENT OF PEPTIDE (1)..(21): fragment of rabbit CETP peptide
RGVVVSSSVAVTFRFPRPDGR

SEQ ID NO: 32 - protein, 21 aa, Rabbit
  FRAGMENT OF PEPTIDE (1)..(21): fragment of rabbit CETP peptide
RPDGREAVAYRFEEDIITTVQ

SEQ ID NO: 33 - protein, 21 aa, Rabbit
  FRAGEMENT OF PEPTIDE (1)..(21): fragment of rabbit CETP peptide
ITTVQASYSQKKLFLHLLDFQ

SEQ ID NO: 34 - protein, 21 aa, Rabbit
  FRAGMENT OF PEPTIDE (1)..(21): fragment of rabbit CETP peptide
LLDFQCVPASGRAGSSANLSV

SEQ ID NO: 35 - protein, 21 aa, Rabbit
  FRAGMENT OF PEPTIDE (1)..(21): fragment of rabbit CETP peptide
ANLSVALRTEAKAVSNLTESR

SEQ ID NO: 36 - protein, 21 aa, Rabbit
  FRAGMENT OF PEPTIDE (1)..(21): fragment of rabbit CETP peptide
LTESRSESLQSSLRSLIATVG

SEQ ID NO: 37 - protein, 21 aa, Rabbit
  FRAGMENT OF PEPTIDE (1)..(21): fragment of rabbit CETP peptide
IATVGIPEVMSRLEVAFTALM

SEQ ID NO: 38 - protein, 21 aa, Rabbit
  FRAGMENT OF PEPTIDE (1)..(21): fragment of rabbit CETP peptide
FTALMNSKGLDLFEIINPEII

SEQ ID NO: 39 - protein, 21 aa, Rabbit
  FRAGMENT OF PEPTIDE (1)..(21): fragment of rabbit CETP peptide
NPEIITLDGCLLLQMDFGFPK

SEQ ID NO: 40 - protein, 16 aa, Rabbit
  FRAGMENT OF PEPTIDE (1)..(16): fragment of rabbit CETP peptide
FGFPKHLLVDFLQSLS

SEQ ID NO: 41 - protein, 31 aa, Artificial
  synthetic hybrid peptide
CQYIKANSKFIGITEFGFPEHLLVDFLQSLS

SEQ ID NO: 42 - protein, 13 aa, Artificial
  synthetic hybrid peptide
XKXVWANTLKAAX

SEQ ID NO: 43 - DNA, 1428 bp, Human
  misc_feature (1)..(1428): nucleotide coding sequence for mature human CETP
tgctccaaaggcacctcgcacgaggcaggcatcgtgtgccgcatcaccaagcctgccctcctggtgttgaaccacgagactgccaaggtgatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagc

SEQ ID NO: 44 - DNA, 1488 bp, Rabbit
  misc_feature (1)..(1488): nucleotide structural coding sequence for mature rabbit CETP
tgtcccaaaggcgcctcctacgaggctggcatcgtgtgtcgcatcaccaagcccgccctcttggtgttgaaccaagagacggccaaggtggtccagacggccttccagcgcgccggctatccggacgtcagcggcgagagggccgtgatgctcctcggccgggtcaagtacgggctgcacaacctccagatcagccacctgtccatcgccagcagccaggtggagctggtggacgccaagaccatcgacgtcgccatccagaacgtgtccgtggtcttcaaggggaccctgaactacagctacacgagtgcctgggggttgggcatcaatcagtctgtcgacttcgagatcgactctgccattgacctccagatcaacacagagctgacctgcgacgctggcagtgtgcgcaccaatgcccccgactgctacctggctttccataaactgctcctgcacctccagggggagcgcgagccggggtggctcaagcagctcttcacaaacttcatctccttcaccctgaagctgattctgaagcgacaggtctgcaatgagatcaacaccatctccaacatcatggctgactttgtccagacgagggccgccagcatcctctcagatggagacatcggggtggacatttccgtgacgggggcccctgtcatcacagccacctacctggagtcccatcacaagggtcacttcacgcacaagaacgtctccgaggccttccccctccgcgccttcccgcccggtcttctgggggactcccgcatgctctacttctggttctccgatcaagtgctcaactccctggccagggccgccttccaggagggccgtctcgtgctcagcctgacaggggatgagttcaagaaagtgctggagacccagggtttcgacaccaaccaggaaatcttccaggagctttccagaggccttcccaccggccaggcccaggtagccgtccactgccttaaggtgcccaagatctcctgccagaaccggggtgtcgtggtgtcttcttccgtcgccgtgacgttccgcttcccccgcccagatggccgagaagctgtggcctacaggtttgaggaggatatcatcaccaccgtccaggcctcctactcccagaaaaagctcttcctacacctcttggatttccagtgcgtgccggccagcggaagggcaggcagctcagcaaatctctccgtggccctcaggactgaggctaaggctgtttccaacctgactgagagccgctccgagtccctgcagagctctctccgctccctgatcgccacggtgggcatcccggaggtcatgtctcggctcgaggtggcgttcacagccctcatgaacagcaaaggcctggacctcttcgaaatcatcaaccccgagattatcactctcgatggctgcctgctgctgcagatggacttcggttttcccaagcacctgctggtggatttcctgcagagcctgagc

SEQ ID NO: 45 - DNA, 63 bp, Rabbit
  misc_feature (1)..(63): nucleotide sequence encoding N-terminal 21 amino acids of human CETP
tgtagcaagggcacctctcacgaggccggcatcgtgtgccggatcaccaaacctgccctcctg

SEQ ID NO: 46 - DNA, 60 bp, Human
  misc_feature (1)..(60): nucleotide sequence encoding amino acids 2-21 of human CETP
agcaagggcacctctcacgaggccggcatcgtgtgccggatcaccaaacctgccctcctg

SEQ ID NO: 47 - DNA, 102 bp, Artificial
  synthetic nucleotide sequence
agcaagggcacctctcacgaggccggcatcgtgtgccggatcaccaaacctgccctcctgcagtacatcaaggccaatagcaagttcatcggcattaccgag

SEQ ID NO: 48 - protein, 13 aa, Artificial
  synthetic peptide
XKXVAAWTLKAAX

SEQ ID NO: 49 - protein, 13 aa, Artificial
  synthetic peptide
AKXVAAWTLKAAA

SEQ ID NO: 50 - protein, 21 aa, Clostridium tetani
  PEPTIDE (1)..(21): 21-amino acid sequence of a universal helper T cell epitope from tetanus toxoid
FNNFTVSFWLRVPKVSASHLE

SEQ ID NO: 51 - protein, 21 aa, Human
  FRAGMENT OF PEPTIDE (1)..(21): fragment of human CETP peptide
CSKGTSHEAGIVCRITKPALL

SEQ ID NO: 52 - protein, 21 aa, Human
  FRAGMENT OF PEPTIDE (1)..(21): fragment of human CETP peptide
KGVVVNSSVMVKFLFPRPDQQ

SEQ ID NO: 53 - protein, 21 aa, Human
  FRAGMENT OF PEPTIDE (1)..(21): fragment of human CETP peptide
NPEIITRDGFLLLQMDFGFPE

SEQ ID NO: 54 - protein, 16 aa, Human
  FRAGMENT OF PEPTIDE (1)..(16): fragment of human CETP peptide
FGFPEHLLVDFLQSLS